(19) United States
(12) Patent Application Publication
(10) Pub. No.: US 2026/0191900 A1
(43) Pub. Date: Jul. 9, 2026

(54) Biofilm Treatment

(71) Applicant: Phytoceutical Limited, Midhurst (GB)

(72) Inventor: William Buchanan, Midhurst (GB)

(73) Assignee: Phytoceutical Limited, Midhurst, WS (GB)

(21) Appl. No.: 19/130,746

(22) PCT Filed: Nov. 20, 2023
(86) PCT No.: PCT/GB2023/053030

§ 371 Date: May 16, 2025

(30) Foreign Application Priority Data
Nov. 18, 2022 (GB) 2217336.3
Aug. 25, 2023 (GB) 2312973.7
Nov. 20, 2023 (WO) PCT/GB2023/053030

(51) Int. Cl. A61K 31/765; A61K 9/00; A61K 9/107; A61K 31/07; A61K 31/785; A61P 17/02; A61P 31/04; B82Y 5/00

(52) U.S. Cl. CPC A61K 31/765; A61K 9/0014; A61K 9/1075; A61K 31/07; A61K 31/785; A61P 17/02; A61P 31/04; B82Y 5/00

(57) ABSTRACT

A compound of Formula (I), or a composition thereof, for use in a method of prevention and/or treatment of a biofilm-related condition. A bio-active agent for use in a method of prevention and/or treatment of a biofilm-related condition, wherein the method comprises administering a compound of Formula (I) or a composition thereof. An article comprising a compound of Formula (I), or a composition thereof. Methods relating to a compound of Formula (I) or a composition thereof.

### FIELD OF THE INVENTION

The present invention relates to the provision of a compound of Formula (I), such as tocopheryl polyethylene glycol succinate (TPGS), and to a composition thereof, for use in the prevention of biofilm formation and/or for use in the prevention or treatment of a biofilm-related condition. The present invention also relates to articles comprising the compound or a composition thereof. The compositions may additionally comprise an antimicrobial agent, such as polyhexamethylene biguanide (PHMB), and/or a retinoid (e.g. retinol).

### BACKGROUND

Biofilms are collections of microorganisms, such as bacteria and/or fungi, within an extracellular matrix that is typically carbohydrate-based. Biofilms typically form on a liquid or solid surface, such as living tissue and/or medical devices.

Cells of a microorganism in a biofilm tend to display different characteristics compared to planktonic cells of the same microorganism. Biofilms provide a microenvironment suitable for sustaining or growing the population of the microorganism, for example by enabling the sharing of nutrients between microorganism cells and/or by providing shelter to cells of the microorganism from conditions that are harmful to the microorganism, such as antimicrobial treatments (e.g. antibiotics and antifungals) and/or immune system response. Cells tend to be more resilient when in a biofilm than when in planktonic form.

Environments favoured for the formation of biofilms tend to be moist and warm. Common examples of parts of the human and/or animal body that typically provide a suitable environment for the formation of biofilms include surfaces within the mouth and sites of infection such as wounds.

The presence of biofilms has been implicated in conditions such as bacterial vaginosis, urinary tract infections, catheter infections, middle-ear infections, formation of dental plaque, gingivitis, eye infections, endocarditis, infections of the lungs, infections in cystic fibrosis, and infections of permanent implants such as joint prostheses, heart valves, and intervertebral discs.

In particular, the ability to control fibroblasts allows the compounds and compositions of the invention to be used to prevent and/or treat fibrosis. Fibrosis may be present in tissues such as the lung, skin, liver, kidney, heart, vascular system, eye, pancreas, intestine, brain and bone marrow. In the mouth, e.g. on teeth, biofilms may manifest as thin films of plaque where they are able to convert sugars and starches into acids that erode tooth enamel. Left untreated, dental plaque hardens to form dental calculus, which is more difficult to remove and can exacerbate further plaque formation.

It has been estimated that 80% of infections in the human body involve biofilms. Furthermore, the use of antibiotics such as β-lactam antibiotics has been found to induce biofilm formation in bacteria such as Staphylococcus aureus (Kaplan, J., et al., mBio. 2012 Jul-Aug; 3(4): e00198-12), hampering conventional treatment with antibiotics.

Wound-related environments for biofilm growth include chronic wounds, venous ulcers and orthopaedic impact wounds.

Left untreated, biofilms in infected areas of the human body, such as wounds, can reduce the efficiency of the immune system response, reduce the efficacy of antimicrobial treatments, prevent the healing of the wound, and lead to an exacerbation of infection. The formation of biofilms can result in ineffective treatment of infected wounds using antimicrobial agents, and increases the opportunity for antibiotic resistance to develop.

Current wound care modalities tend to rely on necrotic cell debridement, surgery, hyperbaric oxygen, antibiotics, nitric oxide, and wound dressing (such as hydrogels, alginates, and silver-impregnated dressing).

Unfortunately, these modalities tend to suffer from various limitations due to lack of specificity and insufficient blood flow at the wound bed. This causes delay in wound contraction, resulting in infection and persistent inflammation.

Additionally, biofilms can form on surfaces other than those in the human or animal body. For example, biofilms can form in healthcare settings such as hospitals, agriculture (e.g. arable farming such as the farming of citrus fruit, grapes, peppers, tomatoes, and/or coffee), aquaculture, water treatment systems, conduits for sewage and/or oil and gas where they can cause clogging, and corrosion heat exchangers where they reduce the efficiency of heat transfer, on marine vessels such as ships, where they cause fouling, and food processing equipment where biofilms can lead to contamination and/or food poisoning.

As such, there is a need for means to prevent biofilm formation, remove a biofilm, and/or to prevent and/or treat biofilm-related conditions. There is also a need for a means to promote secondary healing of tissue (e.g. skin), for example damaged tissue (e.g. skin).

### SUMMARY OF INVENTION

According to a first aspect the invention provides a compound of Formula (I), for example tocopheryl polyethylene glycol succinate (TPGS), or a composition thereof, for use in a method of prevention and/or treatment of a biofilm-related condition.

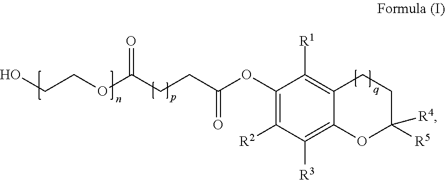

wherein R¹, R², R³ and R⁴ are each independently H or a C1-6 hydrocarbon group that is optionally substituted by one or more Y group; R⁵ is a C6-30 hydrocarbon group; n is from 3 to 100; p is from 0 to 6; q is from 0 to 3; and Y is selected from the list consisting of ether, ester, sulfone, sulfoxide, amide, amine e.g. secondary amine and tertiary amine), boronate ester, ketone and aldehyde.

It has surprisingly been discovered that compounds of Formula (I), such as TPGS, are able to prevent and degrade biofilms. In a medical application, this can make the microorganism in the biofilm less resilient to conventional antimicrobial treatments and/or immune response.

This ability to prevent and/or degrade biofilms, as evidenced by the Examples, is unexpected as no previous art has described or suggested the use of a compound of Formula (I) for this purpose.

Hadinoto. K et al, Eur J Pharm Biopharm, 85(3), pp. 427-443, (2013) discusses the use of TPGS as a stabiliser in combination with other features, may be used within an antibiotic-loaded liquid polymer hybrid nanoparticle. However, TPGS is merely used as a stabiliser, whereas the present inventors have surprisingly discovered that micellar TPGS can provide the benefits discussed herein. Wan, F et al, ACS App Mater Interfaces, 12(1), pp. 380-389, (2020) discusses the use of TPGS to deliver antibiotics in the lungs.

However, TPGS micelles of the present invention are used primarily in relation to topical applications and in related medical devices, such as wound dressings.

Neither of these documents mention the combination of TPGS with bio-active agents such as retinoids (e.g. retinol). Neither of these documents focus on topical administration to the skin of a subject. Indeed, administration to the skin is far more challenging than administration to the lungs, where there is a high surface area and mucous membranes to aid absorption.

In preferred embodiments of the composition of the claimed invention:
the composition further includes an antiseptic such as PHMB;
the compound of formula (I) is in the form of micelles (e.g. with a size of up to 100 nm, such as up to 30 nm);
the composition further includes a retinoid, such as retinol;
the composition is for topical application to a tissue, for example the skin, or a wound of the tissue such as a skin wound;
the composition is in the form of a gel and/or for use as a wound dressing;
the composition includes the compound of formula (I) in an amount of up to 10 wt %, and has a micelle size of up to 100 nm (such as up to 30 nm), and optionally includes PHMB in an amount of up to 0.1 wt % and/or a retinoid in an amount of up to 0.3 wt %; and/or
the composition is a liquid or a gel.

In particular, it has been noted that both Gram-positive and Gram-negative bacteria can be treated by compounds of Formula (I), such as TPGS, and compositions thereof, optionally in combination with an antimicrobial agent such as PHMB. Therefore, compounds of Formula (I), such as TPGS, and compositions thereof can provide broad spectrum treatment of biofilms.

It has also surprisingly been discovered that compounds of Formula (I), such as TPGS, are able to assist the closure of wounds in a three-dimensional tissue model. Specifically, the model showed that compositions including TPGS were able to assist the closure of wounds at depths of 30 μm and 50 μm, indicating improved healing in the epidermis (an example of epithelium) and dermis respectively. Therefore, the present invention can be used to support the healing of tissues, such as skin and epithelium. For instance, the invention may be used to support the healing of a catheter-induced urinary tract infection (UTI).

The composition may comprise an aqueous solution of the compound of Formula (I), such as TPGS. The composition may comprise (e.g. aqueous) micelles of the compound of Formula (I), such as TPGS. The micelles may comprise an aqueous outer phase that comprises the compound of Formula (I) and that encapsulates a hydrophobic inner phase. The hydrophobic phase may contain one or more bio-active agents.

The inventors have discovered that a composition comprising a compound of Formula (I), such as TPGS, and a bio-active therapeutic agent (e.g. two or more bio-active therapeutic agents), in particular an oil-soluble active agent (for example a retinoid (e.g. retinol) and/or curcumin), can also enable the successful reduction in prevalence of bacterial formation of biofilms.

Polyhexamethylene biguanide (PHMB) is a known antimicrobial (antiseptic) agent. However, antimicrobial agents such as PHMB have been shown to be ineffective for treating biofilms (e.g. P. Phillips, et al., Int Wound J, 10(1), pp. 48-55 (2013) and N. Kamaruzzaman et al., Frontiers in Microbiology, Original Research, (2017), 8:1518).

It has surprisingly been determined that a composition comprising both a compound of Formula (I), e.g. TPGS, and an antimicrobial agent, such as PHMB, is significantly more effective than the antimicrobial agent alone. The combination of these agents may be described as providing a synergistic effect. As such, the composition may comprise an antimicrobial agent, such as PHMB.

Indeed, it has been found that the inclusion of antimicrobial agents such as PHMB in compositions of the invention can stabilise retinoids (e.g. retinol) in such compositions (e.g. from oxidation).

The biofilm-related condition may be selected from the list consisting of: dental plaque, dental calculus, dental decay, periodontal disease, gingivitis, inflammatory bowel disease, colorectal disease, peritonitis, urinary tract infection, bacterial vaginosis, vulvovaginal candidiasis, ear infection (such as otitis media), cholesteatoma, sinusitis, adenotonsillitis, prostatitis, pneumonia, bronchitis, COPD, cystic fibrosis, dry eye, blepharitis syndrome, endophthalmitis, keratitis, scleral buckle infection, lacrimal system infection, periorbital infection, endocarditis, myocardial infarction, kidney stones, osteomyelitis, ulcers, acne, psoriasis, hindradenitis suppurativa, atopic dermatitis, candidiasis, onychomycosis, wound (e.g. chronic and/or non-healing wound, orthopaedic impact wound, cutaneous wound, epithelial wound), necrotising fasciitis and infection (e.g. bacterial infection). Additionally or alternatively, the condition may be an orthopaedic wound or a surgical wound. The condition may be a disease.

Surprisingly, it has been found by the present inventors that the composition comprising a compound of Formula (I), e.g., TPGS, is not significantly toxic to skin cells, in contrast to other similar compounds.

A further surprising discovery is that when a topical composition of aqueous micelles of a compound of Formula (I) are provided with a bio-active agent, this enables a more effective delivery of the bio-active agent to the dermis layer (comprising fibroblasts) than if just the bio-active agent is applied topically. This is beneficial because the bio-active agent is able to promote secondary healing. Secondary healing, also known as secondary wound healing, refers to an open wound healing (i.e., a wound that is not stitched closed), The compounds of Formula (I) may promote the delivery of a bio-active agent, such as a bio-active agent (e.g. a retinoid such as retinol), to the dermis layer of the skin. This promotes healthy fibroblast activity through the secretion of collagen proteins. As collagen proteins that help maintain the structural framework of tissues, this aids the secondary healing process. The compounds of Formula (I) may therefore promote a reduction in scarring of a wound (thereby improving the appearance of scar tissue e.g. prevention and/or treatment of scar tissue).

Particular benefits have been observed for compositions that contain the compound of Formula (I) (e.g. TPGS) in an amount of from 5 to 15 wt % and a bio-active agent (e.g. a retinoid such as retinol) in an amount of from 0.1 to 2 wt %. For example, the composition may contain the compound of Formula (I) (e.g. TPGS) in an amount of 10 wt % and a bio-active agent (e.g. a retinoid such as retinol) in an amount of from 0.3 wt % The claimed invention allows for the body's immune system and related self-repair systems to more successfully prevent and/or treat the biofilm-related condition.

Thus, the claimed invention can reduce the instances in which antimicrobial agents, such as antibiotics and/or antifungal agents, need to be administered. This can reduce the chance of antimicrobial resistance developing.

In instances where antimicrobial agents do need to be administered, the claimed invention allows these agents to have a greater efficacy. This can also reduce the chance of antimicrobial resistance developing.

Furthermore, the claimed invention may allow for a lower amount of antimicrobial agent to be used to prevent and/or treat a given biofilm-related condition in order for the same successful prevention and/or treatment outcome to be achieved. This can additionally reduce the chance of antimicrobial resistance developing.

The claimed invention may also allow for a more specific, less broad spectrum antimicrobial agent to be used to prevent and/or treat a given biofilm-related condition in order for the same successful prevention and/or treatment outcome to be achieved. This can also reduce the chance of antimicrobial resistance developing.

Consequently, the present invention can provide significant benefits in terms of the prevention and/or treatment of biofilm-related conditions and in the prevention of antimicrobial resistance developing.

The composition comprising the compound of Formula (I), such as TPGS, may optionally include a bio-active agent, e.g. an antimicrobial agent. Alternatively or additionally, the method of prevention and/or treatment of the biofilm-related condition comprises administering a bio-active agent, e.g. an antimicrobial agent.

According to a second aspect the present invention provides a bio-active agent (e.g. an antimicrobial agent) for use in a method of prevention and/or treatment of a biofilm-related condition, wherein the method comprises administering a compound of Formula (I), such as TPGS, or a composition thereof.

The antimicrobial agent may be an antifungal agent and/or an antibiotic agent. Suitable antibiotic agents include β-lactam antibiotic agent, such as methicillin, ampicillin, amoxicillin, or cloxacillin.

Compounds of Formula (I), such as TPGS, and compositions thereof also have applications in relation to the prevention of the formation of biofilms on articles, such as medical devices.

Thus, according to a third aspect, the invention provides an article comprising a compound of Formula (I), such as TPGS, or a composition thereof. The article may be a medical device. This may be a device that will come into direct contact with the human or animal body, such as a surgical instrument or an implant or prosthesis. This may alternatively be a medical device that will not directly come into contact with the inside of the human or animal body. The medical device may be a catheter, an implant, or a wound dressing.

In particular, the article of the present invention provides particular benefits as a wound dressing. This can provide the benefits mentioned above, such as biofilm clearance, secondary healing effects (scar tissue prevention/treatment), and deep penetration of bio-active agents, in addition to keeping the wound moist to aid natural healing of a wound. The wound dressing may be a gel-based wound dressing.

The gel formulation may be selected such that the viscosity of the composition is increased (e.g. where the viscosity is 10,000 mPas or more at 25° C.). This can provide wound protection. This can provide slow release of a bio-active agent. This may be embedded within or applied onto a wound.

The gel formulation may be selected such that the viscosity of the composition is decreased (e.g. less than 10,000 mPas at 25° C.). This can provide effective wound hydration. Such a formulation may be applied more frequently than a gel formulation of higher viscosity.

The article may suitably comprise the compound of Formula (I), such as TPGS, or the composition thereof on an external surface of the article, for example as a coating of the article.

When the article comprises the compound of Formula (I) or composition thereof, biofilm-forming microorganisms are less likely to form biofilms upon the surface of the article.

The compound of Formula (I), such as TPGS, or composition thereof may be applied to a surface in order to clean (e.g. disinfect) the surface.

Thus, according to a fourth aspect, the present invention provides a method comprising the steps of: providing a compound of Formula (I), such as TPGS, or a composition thereof; and applying the compound or the composition thereof to a surface. The surface may be a surface of an article, such as a medical device. The method may additionally include undertaking a mechanical action to the surface, such as rubbing and/or abrading the surface. The whole of the surface or only part of the surface may be treated. The surface may be exposed to the compound or composition thereof, for example with mechanical action, for a period of 10 seconds or more, or 10 minutes or more, such as 1 hour or more, or 12 hours or more, such as 30 days or less, or 10 days or less. The compound or composition may subsequently be removed from the surface, for example by wiping and/or washing the compound or composition from the surface.

It has been found that compositions comprising a compound of Formula (I), such as TPGS, and one or more bio-active agents can be prepared that are more uniform in particle size (e.g. average particle diameter, therefore having a lower dispersity) and that have improved stability compared to other compositions.

Thus, according to a fifth aspect, the present invention provides a method of providing a micellar composition, the method comprising:
a) providing a compound of Formula (I), such as TPGS, in an aqueous solvent
b) providing a bio-active agent (e.g. a retinoid such as retinol) in an organic solvent; and
c) combining the solution of the compound of Formula (I) with the solution of the bio-active agent to provide a combined sample; and one or both of
   a. subjecting the combined sample to homogenisation whilst the combined sample is at a temperature of from 30° C. to 90° C.; and
   b. diluting the combined sample with an aqueous solvent.

Homogenising the combined sample at an elevated temperature of from 30° C. to 90° C. has been found to provide micelles with a more uniform particle size and that has improved stability compared to other compositions. Dilution has been found to be an effective means to reduce the concentration of components of the composition, such as the organic solvent, to within acceptable limits, and can avoid the requirement to remove such components using tangential flow filtration and/or in vacuo, which can in some cases cause bio-active agents such as retinol to degrade.

The composition containing micelles of the compound of Formula (I), e.g. TPGS, may be prepared by the method of the fifth aspect.

The present disclosure also provides a method of prevention and/or treatment comprising administering a compound of Formula (I) or a composition thereof; a compound of formula (I) or a composition thereof for use as a medicament; and/or a pharmaceutical composition comprising a compound of formula (I).

The compound and compositions thereof will find application in cosmetic uses for example due to their ability to make bio-active agents penetrate deeper into the skin and/or improve the appearance of the skin.

According to a seventh aspect the present invention provides a method of cosmetic prevention and/or treatment, wherein the method comprises:
providing a compound of formula (I) or a composition thereof, and
applying the compound or the composition thereof to the skin of a user in order to enhance the appearance of the skin.

A composition suitable for use in the sixth aspect may be formulated as a cosmetic formulation. The cosmetic formulation may enhance the appearance of the skin by reducing the appearance of scar tissue and/or stretch marks, reducing the number or severity of wrinkles and/or fine lines, improving skin elasticity, and/or by making skin tone more even (e.g. by reducing the appearance of age spots or other conditions relating to excess melanin or hyperpigmentation). This may be a formulation selected from the list consisting of: cream, lotion, gel, semi-solid, dispersion, suspension, foam, mousse and spray. Cosmetic formulations may be formulated in similar manners to the other compositions described herein.

The present application provides the subject-matter of the following clauses:
1. A compound of Formula (I), or a composition thereof, for use in a method of prevention and/or treatment of a biofilm-related condition

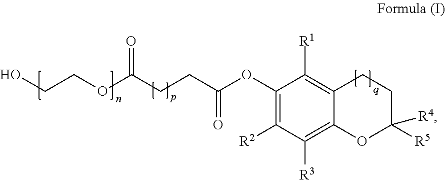

wherein: R¹, R², R³ and R⁴ are each independently H or a C1-6 hydrocarbon group that is optionally substituted by one or more Y group; R⁵ is a C6-30 hydrocarbon group; n is from 3 to 100; p is from 0 to 6; q is from 0 to 3; and Y is selected from the list consisting of ether, ester, sulfone, sulfoxide, amide, amine, boronate ester, ketone and aldehyde.
2. The composition for use of clause 1, wherein the composition comprises aqueous micelles of the compound of Formula (I).
3. The composition for use of clause 2, wherein the micelles have an average diameter of from 1 to 100 nm.
4. The composition for use of any preceding clause, wherein the composition comprises a bio-active agent.
5. The composition for use of clause 4, wherein the bio-active agent is an antimicrobial agent.
6. The composition for use of clause 5, wherein the antimicrobial agent is an antifungal agent and/or an antibiotic agent.
7. The composition for use of clause 6, wherein the antibiotic agent is a β-lactam, a phenol, a diguanide, a quinoline, an alcohol, a peroxide, iodine, octenidine dihydrochloride, a quaternary ammonium salt, or polyhexamethylene biguanide.
8. The composition for use of clause 7, wherein the antimicrobial agent is polyhexamethylene biguanide.
9. The composition for use of clause 4, wherein the bio-active agent is retinol and/or curcumin.
10. The composition for use of any preceding clause, wherein the composition comprises the compound of Formula (I) in an amount of 5% or more by weight.
11. The compound, or composition thereof, for use of any preceding clause, wherein the biofilm-related condition is selected from the list consisting of: dental plaque, dental calculus, dental decay, periodontal disease, gingivitis, inflammatory bowel disease, colorectal disease, peritonitis, urinary tract infection, bacterial vaginosis, vulvovaginal candidiasis, ear infection, cholesteatoma, sinusitis, adenotonsillitis, prostatitis, pneumonia, bronchitis, cystic fibrosis, dry eye, blepharitis syndrome, endophthalmitis, keratitis, scleral buckle infection, lacrimal system infection, periorbital infection, endocarditis, myocardial infarction, kidney stones, osteomyelitis, ulcers, acne, psoriasis, hindradenitis suppurativa, atopic dermatitis, candidiasis, onychomycosis, wound, necrotising fasciitis and infection.
12. The compound, or composition thereof, for use of any preceding clause, wherein:
   R¹, R², R³ and R⁴ are each independently H or a C1-3 alkyl group; R⁵ is a C6-30 hydrocarbon group; n is from 10 to 40; p is from 0 to 2; and q is 0 or 1.
13. The compound, or composition thereof, for use of any preceding clause, wherein Formula (I) is represented by Formula (II):

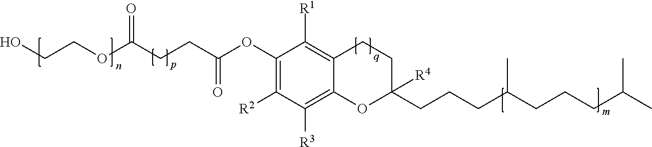

Formula (II), wherein m of Formula (II) is an integer from 0 to 4.
14. The compound, or composition thereof, for use of any preceding clause, wherein the compound is tocopheryl polyethylene glycol succinate.
15. A bio-active agent for use in a method of prevention and/or treatment of a biofilm-related condition, wherein the method comprises administering a compound of Formula (I) or a composition thereof.
16. An article comprising a compound of Formula (I), or a composition thereof.
17. The article of clause 16, wherein the article is a medical device.
18. The medical device is a catheter, an implant, or a wound dressing.
19. A method comprising the steps of: providing a compound of Formula (I) or a composition thereof; and applying the compound or the composition thereof to a surface.

### DETAILED DESCRIPTION

### Compounds of Formula (I), Such as TPGS

The present invention relates to compounds of Formula (I):

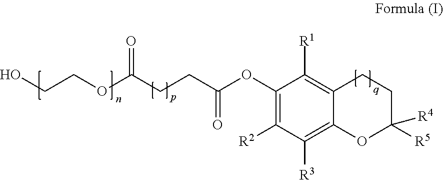

wherein R¹, R², R³ and R⁴ are each independently H or a C1-6 hydrocarbon group that is optionally substituted by one or more Y group; R⁵ is a C6-30 hydrocarbon group; n is from 3 to 100; p is from 0 to 6; q is from 0 to 3; and Y is selected from the list consisting of ether, ester, sulfone, sulfoxide, amide, amine e.g. secondary amine and tertiary amine), boronate ester, ketone and aldehyde.

Hydrocarbon groups may include alkyl, alkenyl, alkynyl and/or aryl groups. Preferably each hydrocarbon group is an alkyl group.

Preferably R¹, R², R³ and R⁴ are each independently H or a C1-3 hydrocarbon group that is optionally substituted by one or more Y group. For example, R¹, R², R³ and R⁴ may each independently be H or a C1-3 hydrocarbon group that is optionally substituted by one Y group; more preferably R¹, R², R³ and R⁴ are each independently be H or a C1-3 hydrocarbon group that is not substituted, for example H or a C1-3 alkyl group. More preferably R¹, R², R³ and R⁴ are each independently a C1, C2 or C3 alkyl group. Most preferably R¹, R², R³ and R⁴ are each C1 (i.e. methyl) groups.

n is from 3 to 100. For example, n may be from 5 to 100, preferably from 10 to 100, or from 15 to 100, such as from 20 to 100. n may be from 5 to 80, such as from 5 to 60, preferably from 5 to 40, such as from 5 to 35, or from 5 to 30, for instance from 5 to 25. n may be from 5 to 60, such as from 10 to 40, or from 15 to 30.

p is from 0 to 6. For example, p may be from 0 to 4, such as from 0 to 3, or from 0 to 2. Preferably p is 0, 1 or 2, most preferably 1.

q is from 0 to 3, such as from 0 to 2. Preferably q is 0 or 1; most preferably q is 1.

Preferably Y is selected from the list consisting of ether, ester, sulfone, sulfoxide, amide, and ketone.

Preferably R⁵ is a C6-26 hydrocarbon group, such as a C6-22 hydrocarbon group, or a C6-18 hydrocarbon group, for example a C6-16 hydrocarbon group. R⁵ may be a C8-30 hydrocarbon group, such as a C10-30 hydrocarbon group, or a C12-30 hydrocarbon group, such as a C14-30 hydrocarbon group or a C16-30 hydrocarbon group. R⁵ may be a C8-26 hydrocarbon group or a C12-20 hydrocarbon group. Preferably the hydrocarbon group of R⁵ is an alkyl group, for example a C8-26 alkyl group or a C12-20 alkyl group.

A compound of Formula (I) may be represented by Formula (II):

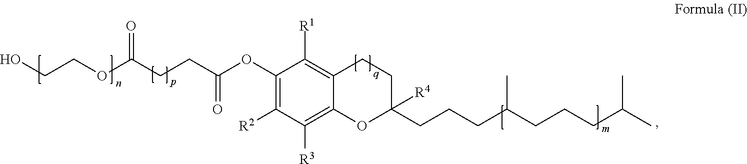

wherein m of Formula (II) is an integer from 0 to 4, preferably from 1 to 3, and most preferably 2.

Options described herein in relation to Formula (I) apply equally to Formula (II) unless inconsistent.

In one embodiment:
the compound is of Formula (I);
R¹, R², R³ and R⁴ are each independently H or a C1-3 alkyl group that is optionally substituted by one Y group;
R⁵ is a C6-30 hydrocarbon group;
n is from 3 to 100;
p is from 0 to 2;
q is 0 or 1; and
Y is selected from the list consisting of ether, ester, sulfone, sulfoxide, amide, amine e.g. secondary amine and tertiary amine), boronate ester, ketone and aldehyde.

In one embodiment:
the compound is of Formula (I);
R¹, R², R³ and R⁴ are each independently H or a C1-3 alkyl group;
R⁵ is a C6-30 hydrocarbon group;
n is from 10 to 40;
p is from 0 to 2; and
q is 0 or 1.

In one embodiment:
the compound is of Formula (II);
R¹, R², R³ and R⁴ are each independently H or a C1-3 alkyl group;
m is from 1 to 3;
n is from 10 to 40;
p is from 0 to 2; and
q is 0 or 1.

In one embodiment:
the compound is of Formula (II);
R¹, R², R³ and R⁴ are each independently C1 or a C2 alkyl group;
m is from 1 to 3;
n is from 15 to 30;
p is 1; and
q is 1.

Most preferably the compound of Formula (I) is tocopheryl polyethylene glycol succinate (TPGS also known as tocofersolan or tocophersolan).

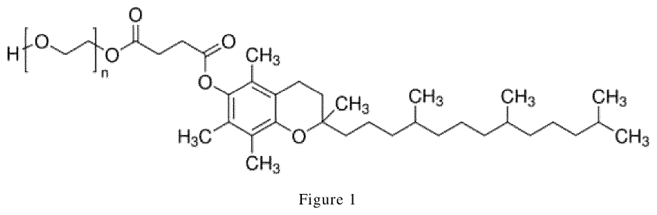

FIG. 1 of the accompanying drawings shows the structure of TPGS. TPGS is a water-soluble vitamin E conjugate. In vivo, enzymatic cleavage of TPGS can provide tocopherol (vitamin E). The compound of Formula (I) may be represented by the compound shown in FIG. 1.

Preferably the polyethylene glycol (PEG) unit, as shown in the brackets of the structures of TPGS, has a number average molecular weight of from 100 to 5000, such as from 500 to 3000, preferably from 800 to 2500, such as 800 to 1500, most preferably 1000. TPGS with a PEG group average (e.g. number average) molecular weight of 1000 g mol⁻¹ is known as “TPGS 1000”, e.g. “d-a-tocopheryl polyethylene glycol 1000 succinate”, and is commercially available (CAS 9002-96-4). TPGS 2000, having a PEG group average molecular weight of 2000 g mol⁻¹ is also commercially available.

The compound of Formula (I), e.g. TPGS, may be present in any stereoisomeric or tautomeric form. Preferably the compound is in the D-α-stereoisomer form, as shown below:

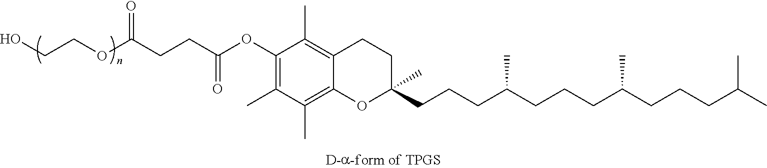

### Composition

It will be appreciated that the formulation of the composition may not be restricted other than as required by the claimed invention.

The composition may be a pharmaceutical composition. In one embodiment the pharmaceutical composition (e.g. formulation) comprises at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

The pharmaceutical compositions can be in any form suitable for oral, parenteral, topical, intranasal, ophthalmic, otic, rectal, intra-vaginal, urethral or transdermal administration. These pharmaceutical compositions may be suitable for administration orally; rectally, for example, by enema, suppository or catheter; or nasally, for example, endoscopically through a nasogastric or nasoduodenal tube. Preferably compositions of the invention are suitable for topical application/administration. Compositions of the invention may be suitable for application to, around and/or within a wound.

To prepare the pharmaceutical compositions of this invention, an effective amount of a compound of the present invention, as the active ingredient is combined in intimate admixture with a pharmaceutically acceptable carrier, which carrier may take a wide variety of forms depending on the form of preparation desired for administration.

For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient, calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The composition may, for example, be in the form of a powder, tablet, liquid (such as a rinse (e.g. mouthwash)), film, cream, lotion, gel (e.g. amorphous gel), semi-solid, dispersion, suspension, foam, mousse, spray, hydrogel, hydrocolloid or paste (e.g. toothpaste). Preferably the composition is a liquid (e.g. mouthwash), cream, lotion, gel (e.g. amorphous gel), dispersion, suspension, foam, mousse, spray or paste (e.g. toothpaste).

The composition may be formulated for inhalation (e.g. by Pulmonary Function Testing (PFT) equipment (e.g. a spirometer), a ventilator, a humidifier, a nebulizer, a positive airway pressure (PAP) device, a manual resuscitator, a mask, an item of oxygen equipment, a circuit, a disposable, a respirator, an inhaler, or an inhalation chamber, a nebuliser, or a part thereof). Preferably compositions for inhalation include only small amounts of PHMB, or do not include PHMB, to avoid fibrosis that can be caused by PHMB inhalation. The amount of PHMB in a composition formulated for inhalation may be 0.1 wt % or less, such as 0.01 wt % or less, or 0.001 wt % or less.

Preferably the composition is combined with hyaluronic acid (HA) to provide a liquid/gel that may be administered to the subject (e.g. a wound thereof).

The composition may be freeze-dried and optionally rehydrated.

The composition may include the compound of Formula (I), e.g. TPGS, in an amount, by weight, of 0.1% or more, such as 1% or more, or 2% or more, or 3% or more, or 5% or more, such as 8% or more, or 10% or more, such as 15% or more, or 20% or more. The amount of TPGS in the composition may be 70% or less, such as 50% or less, or 40% or less, or 30% or less, for example 25% or less, or 20% or less, such as 15% or less, or 12% or less, such as 10% or less. For example, the amount of TPGS in the composition may be from 0.1% to 70% by weight, such as from 1% to 50%, or from 5% to 30%, for example 5% to 10% by weight.

The TPGS can be dissolved in aqueous media and non-aqueous media (e.g. oils, lipophilic media); preferably the TPGS is in an aqueous solution.

Compounds of Formula (I), such as TPGS, are surfactants and can form micelles. In aqueous media, each micelle typically has a hydrophobic inner phase, which is suitable for containing a bio-active agent, and a hydrophilic outer phase, which may include excess TPGS. However, it may be that the composition comprises micelles with hydrophilic inner phases and hydrophobic outer phases. The composition may comprise multi-layered micelles, such as hydrophobic-hydrophilic-hydrophobic micelles, or hydrophilic-hydrophobic-hydrophilic micelles.

WO 2017/194965 A1 (which is incorporated herein by reference) discloses compositions and cosmetic formulations comprising micelles of TPGS, and describes the use of the compositions and formulations in cosmetic applications, to deliver bio-active therapeutic agents. However, this document does not disclose or teach towards the use of TPGS in relation to the prevention and/or treatment of biofilm-related conditions, or other applications relating to microorganisms, especially biofilms.

The water phase of the composition may optionally be evaporated. The micelles may optionally be rehydrated.

The average (e.g. mean) diameter of the micelles (hydrodynamic particle size) may be measured by dynamic light scattering (DLS). The micelles may have an average diameter of 100 nm or less, or 60 nm or less, or 45 nm or less, 40 nm or less, 35 nm or less, preferably 30 nm or less, or 25 nm or less, 24 nm or less, 23 nm or less, 22 nm or less, 21 nm or less, or 20 nm or less. The micelles may have an average diameter of 1 nm or more, such as 2 nm or more, or 4 nm or more, preferably 8 nm or more, 10 nm or more, or 12 nm or more. The micelles may have an average (e.g. mean) diameter of from 1 to 100 nm, such as from 2 to 60 nm, or from 4 to 40 nm, or preferably from 8 to 30 nm, or from 8 to 25 nm. The micelles may have an average (e.g. mean) diameter of from 8 to 16 nm.

Size distribution may be described with reference to volume, intensity or number. In the present specification “average diameter” refers to a volume average diameter, unless otherwise stated.

The micelles have been found to display low dispersity. The polydispersity index (PDI) of the micelles may be 0.5 or less, such as 0.2 or less, or 0.15 or less, preferably 0.12 or less, or 0.11 or less, such as 0.1 or less. The polydispersity index of the micelles may be 0.0001 or more, such as 0.0005 or more, or 0.001 or more, preferably 0.005 or more, such as 0.01 or more, such as 0.02 or more, or 0.04 or more. The polydispersity index of the micelles may be from 0.0001 to 0.5, such as from 0.005 to 0.12. PDI may be determined by dynamic light scattering (DLS).

The micelles may have an average diameter of 100 nm or less and a PDI of 0.5 or less. The micelles may have an average diameter of from 1 to 100 nm and a PDI of from 0.0001 to 0.5. The micelles may have an average diameter of from 8 to 30 nm and a PDI of from 0.005 to 0.12.

The average diameter may refer to the empty or unloaded micelles, i.e. without cargo, or to loaded micelles, i.e. with cargo.

The composition containing micelles of the compound of Formula (I), e.g. TPGS, may be prepared by a method comprising:
a) providing an organic solvent (e.g. ethanol) and optionally dissolving an oil-soluble active agent and/or a surfactant such as polysorbate in the organic solvent to form a hydrophobic phase; and
b) adding the hydrophobic phase into an aqueous solution of the compound of Formula (I);
c) and optionally removing a portion of the organic solvent.

According to the fifth aspect, the present invention provides a method of providing a micellar composition, the method comprising:
a) providing a compound of Formula (I), such as TPGS, in an aqueous solvent
b) providing a bio-active agent (e.g. a retinoid such as retinol) in an organic solvent;
c) combining the solution of the compound of Formula (I) with the solution of the bio-active agent to provide a combined sample; and
d) subjecting the combined sample to homogenisation whilst the combined sample is at a temperature of from 30° C. to 90° C.

The composition containing micelles of the compound of Formula (I), e.g. TPGS, may be prepared by the method of the fifth aspect.

The aqueous solvent includes water. The organic solvent is preferably a water-miscible organic solvent, especially an alcohol such as ethanol. The aqueous solvent may comprise the compound of Formula (I) in an amount of 0.1% or more, such as 1% or more, or 10% or more by weight. The aqueous solvent may comprise the compound of Formula (I) in an amount of 50% or less, such as 35% or less, or 25% or less by weight. The aqueous solvent may comprise the compound of Formula (I) in an amount of from 0.1 to 50% by weight, for example from 1 to 25% by weight. The organic solvent may comprise the bio-active agent (e.g. a retinoid such as retinol) in an amount of 0.01% or more, such as 0.05% or more, or 0.1% or more, such as 0.4% or more by weight. The organic solvent may comprise the bio-active agent (e.g. retinol) in an amount of 20% or less, such as 10% or less, or 5% or less, for example 2% or less or 1% or less by weight. The organic solvent may comprise the bio-active agent (e.g. retinol) in an amount of from 0.01% to 20%, for example from 0.05% to 5% by weight.

Contacting the solutions may comprise mixing the solutions, for example to form a mixture. Homogenisation may be performed using a homogeniser, for example a Fisher brand 850 Homogeniser. The homogeniser may be set to a rotation speed of 1000 rpm or more, such as 3000 rpm or more, such as 4500 rpm or more, such as 10,000 rpm or less, or 6,000 rpm or less, for example from 1000 to 10,000 rpm. Preferably the temperature of the combined sample during homogenisation is from 40° C. to 90° C., such as from 45° C. to 90° C. The temperature of the combined sample during homogenisation may be from 30° C. to 80° C., or from 30° C. to 70° C., such as from 30° C. to 60° C., or from 30° C. to 55° C. The temperature may be from 40° C. to 70° C., such as from 40° C. to 60° C., or from 45° C. to 55° C.

Homogenisation may be performed for a period of time of 1 minute or more, such as 5 minutes or more, or 10 minutes, or more, such as 20 minutes or more. The period of time may be 48 hours or less, such as 6 hours or less, or 1 hour or less, for example from 1 minute to 48 hours or from 10 minutes to 6 hours. Homogenisation may be performed until 20% or more of the organic solvent (by volume) has been removed, such as 50% or more, or 70% or more, preferably 80% or more, such as 90% or more, or 95% or more, or 98% or more, such as until 99% or more of the organic solvent has been removed.

The homogenised sample may be allowed to cool after homogenisation, for example to room temperature.

The method may comprise combining the homogenised sample with a solution of a second bio-active agent (e.g. PHMB). The solution may comprise the second bio-active agent in an amount of 0.001% or more, such as 0.01% or more, or 0.05% or more, such as 0.08% or more by weight. The solution may comprise the bio-active agent (e.g. PHMB) in an amount of 20% or less, such as 10% or less, or 5% or less, for example 2% or less or 1% or less, such as 0.5% or less, or 0.3% or less by weight. The solution may comprise the bio-active agent (e.g. PHMB) in an amount of from 0.01% to 20%, for example from 0.05% to 2% by weight. The solution may comprise an organic solvent (e.g. ethanol) and/or an aqueous solvent (e.g. water).

Organic solvents, such as water-miscible organic solvents, may need to be removed and/or diluted to yield the micellar composition. The removal of organic solvents can typically be performed using distillation (e.g. vacuum distillation), warming, tangential flow filtration (TFF) or a combination thereof. Preferably dilution is used in combination with another method of removal of the organic solvent. Surfactants such as polysorbate may not need to be removed in order to provide the micellar composition. However, some or all polysorbate can be removed by using complexation (e.g. with cobalt and thiocyanate) or reverse filtration. Compositions may be diluted to ensure that components of the composition are within an acceptable (e.g. cosmetically or pharmaceutically acceptable) concentration range.

Homogenization may be required to promote micellar formulation. Such homogenizers may include pressure homogenizers, mechanical homogenizers, and/or ultrasonic homogenizers. Preferably, the homogenizer will be a mechanical homogenizer.

The amount of hydrophobic phase that is added into the aqueous solution of the compound of Formula (I) may be 1% or more by volume of the aqueous TPGS, such as 2% or more, 5% or more, preferably 8% or more, 10% or more, 12% or more, or 15% or more, such as 18% or more. The amount of hydrophobic phase that is added into the aqueous solution of the compound of Formula (I) may be 50% or less by volume of the aqueous TPGS, such as 40% or less, 35% or more, preferably 30% or less, 28% or less, 25% or less, or 22% or less, such as 20% or less. Preferably, the amount of hydrophobic phase that is added into the aqueous solution of the compound of Formula (I) is from 1% to 20% by volume of the aqueous solution of the compound of Formula (I). The amount of hydrophobic phase may be in the range 2% to 19%, 3% to 18%, 4% to 17%, 5% to 16%, 6% to 15%, 7% to 14%, 8% to 13%, 9% to 12%, 2% to 20%, 3% to 20%, 4% to 20%, 6% to 20%, 7% to 20%, 8% to 20%, 9% to 20%, 10% to 20%, 11% to 20%, 12% to 20%, 13% to 20%, 14% to 20%, 15% to 20%, 16% to 20%, 17% to 20%, 18% to 20%, 19%, 19.5% or 20% by volume of the aqueous solution of the compound of Formula (I).

The composition may comprise one or more bio-active agents.

The bio-active agent may enhance secondary healing that can be achieved with the composition. When used in topical applications, the bio-active agent may be selected such that imparts secondary healing to particular skin tissues, such as the epidermis, the dermis, and/or the hypodermis. In particular, the main cellular component of the epidermis targeted in secondary healing are the keratinocytes. In particular, the main cellular component of the dermis targeted in secondary healing are fibroblasts.

The bio-active agent may be an antimicrobial (antiseptic) agent. The antimicrobial agent may be an antifungal agent and/or an antibiotic agent. Suitable antibiotic agents include β-lactam antibiotic agent (such as methicillin, ampicillin, amoxicillin, or cloxacillin), a phenol (e.g. triclosan, hexachlorophene, chlorocresol, or chloroxylenol), diguanide (e.g. chlorhexidine gluconate), a quinoline (such as hydroxyquinolone, dequalium chloride, and chlorquinaldol), an alcohol (such as ethanol or isopropanol), a peroxide (such as hydrogen peroxide or benzoyl peroxide), iodine, octenidine dihydrochloride, a quaternary ammonium salt (such as benzalkonium chloride, cetylpyridinium chloride, or cetrimide), or PHMB. Preferably the antimicrobial agent is PHMB. In one embodiment the composition does not contain PHMB, or another antiseptic agent. The activity of PHMB can be significantly reduced by exposure to (e.g. washing with) an inorganic salt solution, such as saline. Therefore, the activity of PHMB can be stopped when desired by exposure to an inorganic salt solution.

Antifungal agents may be selected from the list consisting of: polyenes (e.g. amphotericin B deoxycholate, liposomal amphotericin B, amphotericin B lipid complex, and nystatin), azoles (e.g. ketoconazole, miconazole, clotrimazole, itraconazole, isavuconazonium sulfate (isavuconazole), fluconazole, voriconazole and Posaconazole), allylamines (e.g. terbinafine), echinocandins (e.g. anidulafungin, caspofungin and micafungin) Mitotic Inhibitors: (e.g. griseofulvin), antimetabolites (e.g. flucytosine, and Ciclopirox), quinoline derivatives (e.g. iodoquinol and clioquinol), potassium iodide (e.g. saturated solution of potassium iodide (SSKI)), and zinc pyrithione. Antifungal agents may be organic (e.g. naturally occurring).

The bio-active agent may be retinol, curcumin, a tocotrienol, a tocopherol, PHMB (e.g. buffered PHMB), insulin, ascorbic acid (e.g. L-ascorbic acid), a flavonoid, a tannin, a phenolic acid, a coumarin, an alkaloid, a cannabinoid (e.g. cannabidiol), a terpenoid, an essential oil, a lectin, a peptide, chitosan or hyaluronic acid. The bio-active agent may be a retinoid (e.g. retinol, retinal (retinaldehyde), tretinoin (retinoic acid), isotretinoin, alitretinoin, etretinate, acitretin, adapalene, bexarotene, tazarotene, trifaroteneor, retinyl esters such as retinyl palmitate, and combinations thereof), curcumin, a tocotrienol, a tocopherol, PHMB (e.g. buffered PHMB), insulin, ascorbic acid (e.g. L-ascorbic acid), a flavonoid, a tannin, a phenolic acid, a coumarin, an alkaloid, a cannabinoid (e.g. cannabidiol), a terpenoid, an essential oil, a lectin, a peptide, chitosan or hyaluronic acid. Preferably the bio-active agent is a retinoid, such as retinol or retinal, more preferably retinol or retinal. The bio-active agent may be two or more, such as three or more retinoids. The bio-active agent may be two or more of the retinoids: retinol, retinaldehyde, retinoic acid, and retinoic acid esters. The bio-active agent may be retinol and retinaldehyde. As retinol can oxidise to retinaldehyde, the bio-active agent may comprise retinol and retinaldehyde and/or retinoic acid, where the retinaldehyde and/or retinoic acid has formed from the oxidation of retinol.

The bio-active agent is preferably PHMB, retinol or curcumin.

The composition preferably contains an antiseptic agent, such as PHMB, in an amount of 0.001% or more, such as 0.002% or more, or 0.005% or more, preferably 0.01% or more, such as 0.05% or more, or 0.08% or more by weight. The composition may contain the antiseptic agent (e.g. PHMB) in an amount of 40% or less by weight, such as 20% or less, or 10% or less, or 5% or less, or 3% or less, preferably 2% or less, or 1.5% or less, such as 1% or less, for example 0.5% or less, or 0.2% or less. The composition may contain the antiseptic agent (e.g. PHMB) in an amount of from 0.001% to 40% by weight, such as from 0.005% to 10%, or from 0.01% to 2% by weight.

The composition preferably contains curcumin in an amount of 0.001% or more, such as 0.002% or more, or 0.005% or more, preferably 0.01% or more, such as 0.05% or more, or 0.08% or more by weight. The composition may contain curcumin in an amount of 40% or less by weight, such as 20% or less, or 10% or less, or 5% or less, or 3% or less, preferably 2% or less, or 1.5% or less, such as 1% or less, for example 0.5% or less, or 0.2% or less. The composition may contain curcumin in an amount of from 0.001% to 40% by weight, such as from 0.005% to 10%, or from 0.01% to 2% by weight.

The composition preferably contains a retinoid (e.g. retinol) in an amount of 0.01% or more by weight, such as 0.05% or more, or 0.1% or more, such as 0.2% or more, or 0.25% or more. The composition may contain a retinoid (e.g. retinol) in an amount of 40% or less by weight, such as 20% or less, or 10% or less, preferably 5% or less, or 3% or less, such as 2% or less, or 1.5% or less, such as 1.2% or less. The composition may contain a retinoid (e.g. retinol) in an amount of from 0.01% to 40% by weight, such as from 0.01% to 10%, or from 0.05% to 5%, or from 0.1% to 5% by weight.

It has been found that retinol is not toxic to fibroblasts at concentrations of 6 μg/mL or less. Preferably the retinoid (e.g. retinol) delivered to the fibroblasts is in an amount of 6 μg/mL or less, such as 5 μg/mL or less, or 4 μg/mL or less, such as 3 μg/mL or less, for example 2.5 μg/mL or less. The retinoid may be included in a concentration of 0.1 μg/mL or more, such as 0.5 μg/mL or more, or 1 μg/mL or more, or 2 μg/mL or more. The concentration of retinoid in the composition may be from 0.1 to 6 μg/mL, for example from 0.5 to 6 μg/mL, or from 0.5 to 3 μg/mL, such as from 0.5 to 2.5 μg/mL.

Preferably the composition comprises the compound of formula (I) in an amount of 0.1 wt % or more, and a bio-active agent (e.g. a retinoid and/or PHMB) in an amount of 0.01 wt % or more. For example, the composition may comprise the compound of formula (I) in an amount of 1 wt % or more and the bio-active agent (e.g. a retinoid, such as retinol) in an amount of 0.05 wt % or more. The composition may comprise the compound of formula (I) in an amount of 5 wt % or more and the bio-active agent (e.g. a retinoid, such as retinol) in an amount of 0.1 wt % or more. The composition may comprise the compound of formula (I) in an amount of from 0.1 to 40 wt % and the bio-active agent (e.g. a retinoid, such as retinol) in an amount of from 0.01 to 10 wt %. The composition may comprise the compound of formula (I) in an amount of from 1 to 20 wt % (e.g. 1 to 10 wt %) and the bio-active agent (e.g. a retinoid, such as retinol) in an amount of from 0.1 to 2 wt %. The composition may comprise the compound of formula (I) in an amount of from 1 to 20 wt % (e.g. 5 to 15 wt %) and the bio-active agent (e.g. a retinoid, such as retinol) in an amount of from 0.1 to 0.8 wt % (e.g. 0.1 to 0.5 wt %). The composition may comprise the compound of formula (I) in an amount of from 1 to 10 wt % and the bio-active agent (e.g. a retinoid, such as retinol) in an amount of from 0.01 to 0.3 wt %. In one embodiment such a composition does not contain PHMB, or another antiseptic agent.

Particular benefits have been observed for compositions that contain the compound of Formula (I) (e.g. TPGS) in an amount of from 5 to 15 wt % and a bio-active agent (e.g. a retinoid such as retinol) in an amount of from 0.1 to 2 wt %.

For example, the composition may comprise the compound of formula (I) (e.g. TPGS) in an amount of 1 wt % or more and the bio-active agent (e.g. an antiseptic such as PHMB) in an amount of 0.001 wt % or more (e.g. 0.005 wt % or more). The composition may comprise the compound of formula (I) in an amount of from 1 to 10 wt % and the bio-active agent (e.g. PHMB) in an amount of from 0.005 to 0.1 wt %. Such compositions may optionally include a retinoid (e.g. retinol) in an amount of 0.01 wt % or more, such as 0.1 wt % or more, or from 0.01 to 2 wt % (e.g. from 0.1 to 0.3 wt %).

Combinations of two or more bio-active agents can be used. The composition may include an antimicrobial agent and one or more agents selected from the list consisting of a retinoid (e.g. retinol), curcumin, a tocotrienol, a tocopherol, insulin, ascorbic acid, a flavonoid, a tannin, a phenolic acid, a coumarin, an alkaloid, a terpenoid, an essential oil, a lectin, a peptide, chitosan or hyaluronic acid. In one preferred embodiment, the composition includes two or more of a retinoid (e.g. retinol), PHMB and curcumin. More preferably the composition includes a retinoid (e.g. retinol) and PHMB.

The composition may include one or more bio-active agents and micelles of the compound of Formula (I), e.g. TPGS. The bio-active agent may be encapsulated within the micelles of the compound of Formula (I).

The composition may contain an antiseptic agent, such as PHMB, in an amount of from 0.001% to 40% and a retinoid (e.g. retinol) in an amount of from 0.01% to 40%. For example, the composition may contain an antiseptic agent, such as PHMB, in an amount of from 0.01% to 2% and a retinoid (e.g. retinol) in an amount of from 0.1% to 5%.

For example, the composition may comprise one or more oil-soluble bio-active agents. These may be encapsulated, as a cargo, in the hydrophobic inner phase of the micelles. Preferably, the oil-soluble bio-active agent is an oil-soluble vitamin. Suitable oil-soluble bio-active agents include a retinoid (e.g. retinol), curcumin, a tocotrienol and/or a tocopherol. One or more oil-soluble bio-active agents may be added to the organic solvent before it is combined (e.g. mixed) with the compound of Formula (I) in the aqueous solvent.

The composition may additionally or alternatively comprise a water-soluble bio-active agent, e.g. in the hydrophilic outer phase of the micelles. Suitable water-soluble bio-active agents include PHMB (e.g. buffered PHMB), insulin, ascorbic acid, hyaluronic acid and/or L-ascorbic acid. Suitable water-soluble bio-active agents include PHMB (e.g. buffered PHMB), insulin, ascorbic acid and/or L-ascorbic acid. Preferably the composition includes PHMB. One or more water-soluble bio-active agents may be added to the aqueous solvent before and/or after the aqueous solvent is combined (e.g. mixed) with the solution of the bio-active agent in the organic solvent.

A range of permissible solvents may be used in conjunction with the methods of the present disclosure to solubilise bio-active agents for encapsulation in micelles depending on the chosen application. Suitable solvents will depend on the bio-active being incorporated into the micellar composition and may be selected accordingly. If it is intended to incorporate one bio-active in the micellar composition, for example a retinoid (e.g. retinol), then an appropriate solvent will be one in which the chosen bio-active is soluble. Depending on the desired application, a mixture of miscible solvents may be used. Similarly, if it is intended to incorporate more than one bio-active in the micellar composition, for example a retinoid (e.g. retinol) and ascorbyl palmitate, then an appropriate solvent will be one in which both of the chosen bio-actives are soluble. The organic solvent may be a volatile organic solvent and or a GRAS solvent. The organic solvent may be selected from the list consisting of diethyl ether, tetrahydrofuran, 2-methyl tetrahydrofuran, dimethylformamide, methanol, N-methyl pyrrolidinone, formic acid, acetic acid, anisole, butyl butyrate, 1,3-butylene glycol, ethanol, ethyl acetate, ethyl benzoate, ethyl butyrate, ethyl decanoate, ethyl formate, ethyl hexanoate, ethyl lactate, ethylene dichloride, glycerol, glyceryl monooleate, glyceryl palmitostearate, isoamyl acetate, isobutyl acetate, isopropyl acetate, isopropyl alcohol, isopropyl citrate, lactic acid, linoleic acid, methyl acetate, octanoic acid, propionic acid, propyl acetate, stearic acid, ethyl vanillin, or limonene. The organic solvent is preferably an alcohol. More preferably the organic solvent is a C1-C6 (e.g. C1-C3) alcohol. The organic solvent is preferably ethanol. The organic solvent may additionally or alternatively comprise polysorbate (e.g. polysorbate 40 and/or polysorbate 80), and/or a poloxamer, such as poloxamer 407. Alternative or additional organic solvents include ethoxylated solubilisers (e.g. Soluplus® (BASF), a polyvinyl caprolactam-polyvinyl acetate-polyethylene glycol graft copolymer, CAS 402932-23-4), polymeric solubilisers and/or matrix forming polymer. Alternative or additional organic solvents include lecithin liquid and lecithin powder.

Preferably, at least a portion of the organic solvent is removed by subjecting the micellar composition to a vacuum, heating, sonification, TFF (e.g. with prior dilution), or a combination thereof. Preferably, the organic solvent is partially or completely removed by subjecting the micellar composition to heating and a vacuum. In methods where heating is applied, either alone or in combination with other methods such as application of a vacuum, preferably, the temperature used to remove the volatile organic solvent will be from 30° C. to 70° C., such as from 30° C. to 50° C.

The micelles may be loaded with an amount of cargo (e.g. the bio-active agent) that is measured as percent by weight relative to the total weight of the TGPS and the cargo. The amount of cargo may depend on the potency or nature of the bio-active agent and/or the application that the composition is to be used in. Generally, the amount may be 0.01% or more, such as 0.1% or more, 0.2% or more, 0.5% or more, 0.8% or more, or preferably 1% or more, 1.2% or more, 1.5% or more, 1.8% or more, or 1.9% or more. The amount may be 50% or less, such as 40% or less, 35% or less, or preferably 30% or less, 25% or less, such as 22% or less, or 20% or less. The amount may be from 0.01 to 50%, from 0.05 to 30%, from 0.05 to 20%, or preferably from 0.1 to 20%, from 0.2 to 20%, or from 0.5 to 20%.

The composition may comprise a thickener and/or a stabiliser. Suitable thickeners and stabilisers include those selected from the list consisting of: xanthan gum; hyaluronic acid; hydroxyethyl cellulose; collagen; collagen peptides; clays (e.g. bentonite/hectorite clays); alginates; cellulose and esters thereof; hydroxypropyl cellulose; methylcellulose; carboxymethylcellulose; gellan gum; polymethacrylates such as glyceryl polymethacrylate; acrylates; starches such as potato starch; chitosan; polyvinyl alcohol; hydroxypropyl methylcellulose; carbomer; and hydrofibre. Preferably the composition comprises xanthan gum, especially when the composition comprises a compound of Formula (I) alone and optionally a retinoid (e.g. retinol), for example when the composition does not comprise PHMB. Preferably the composition comprises hydroxyethyl cellulose, especially where PHMB is used with a compound of Formula (I) (and optionally retinol). Other preferable thickeners and/or stabilisers include hydrogel. The hydrogel may be sourced from a natural/biological source, for example jellyfish (i.e. jellyfish collagen).

Retinol can conventionally be provided as a mixture with additives such as alkanes (e.g. undecane and tridecane), tocopherol and/or polysorbate. These additives may enhance the stability of the retinol (e.g. to prevent oxidation). A composition of the invention may include one or more of these additives in an amount of 5 wt % or less, such as 2 wt % or less, or 1 wt % or less, for example 0.5 wt % or less, or 0.3 wt % or less. A composition of the invention may include one or more of these additives in an amount of 0.0001 wt % or more, such as 0.001 wt % or more, or 0.01 wt % or more, for example from 0.0001 wt % to 5 wt %, or from 0.001 wt % to 0.5 wt %.

### Article

The TPGS or composition thereof may be coated and/or fixed (e.g. adhered) to materials. Techniques that may be used to apply the TPGS or composition thereof to a surface include 3D printing, electrospinning, electrophoretic deposition, dip-coating, drop casting, sol-gel deposition, biomimetic deposition, plasma spraying, layer-by-layer deposition, physical vapour deposition, anodization (e.g. K. Kravanja et al., Materials & Design 217 (2022) 110653), hydrogel coating techniques (e.g. S. Bohara et al., Biomaterials Research (2022) 26:26), amphiphilic polymeric coating (e.g. W. Xi et al., Nat. Comm. (2021) 12:5473), leucocyte- and platelet-rich fibrin products such as fibronectin/vitronectin exudate (e.g. M. Lollobrigida et al., BioMed Res. Int., (2018):9031435) and/or nanocoating (e.g. Ming-qi Chen, Front. Bioeng. Biotechnol. (2022) 10:878257).

The composition may be adhered to a surface such that over time the encapsulated bio-active may release. Modified or controlled release (e.g. slow release) may, for example, be achieved through entrapment methods of nano- and micro-encapsulation (e.g. K. Kravanja et al., Materials & Design 217 (2022) 110653), implantable drug delivery devices (e.g. S. Stewart et al., Polymers (2018), 10, 1379), tissue adhesives (e.g. B. Mizrahi et al., Stud Mechanobiol Tissue Eng Biomater (2011) 8: 39-56). Modified or controlled release (e.g. slow release) may be achieved using the methods described in E. Gamez-Herrera et al., Eur J. of Pharmaceutics and Biopharmaceutics Volume 152, July 2020, Pages 327-339 (asymmetric electrospun nanofibers decorated with electrosprayed poly(lactic-co-glycolic acid) microparticles); I. Macha et al. S N Applied Sciences 2:176 (2020) (cotton handwoven fabric hydroxyapatite polylactic acid composites); H. Sandoz et al., Wounds international. “Biofilm-based wound care with cadexomer iodine” URL: https://www.woundsinternational.com/download/resource/6097 Accessed 18 Nov. 2022 (cadexomer iodine-based wound dressings); J. Yuan et al. Biomater. Sci., 2020, 8, 5647-5655 (scaffolds made of silk fibroin nanofibers and chitosan); SynBiosys brochure 2022—Innocore Pharmaceuticals URL: https://www.innocorepharma.com/content/finder-upload/files/synbiosys-brochure-2022.pdf accessed 18 Nov. 2022 (SynBiosys® biodegradable polymeric drug delivery system comprising poly(ether-ester) multiblock copolymers); Ingell factsheet—Innocore Pharmaceuticals URL: https://www.innocorepharma.com/content/finder-upload/files/Ingell %20factsheet(2).pdf accessed 18 Nov. 2022 (PCLA-PEG-PCLA tri-block copolymers with aliphatic end groups); L. Zheng et al. (2020) Front. Bioeng. Biotechnol. 8:593768 (e.g. biopolymers such as bacterial cellulose); T. Abdullah et al. Scientific Reports 10: 20428 (2020) (polycaprolactone coated chitin-lignin gel fibrous scaffolds).

The invention also provides an article having a surface that is made of polymer, or that has a polymer coating.

Thus the TPGS or composition thereof may be included in, impregnated within, or coated upon a material, such as a plastic, a metal, wood, and/or fabric. The material may be used to form all or part of the article. Preferably the material, is used to form at least part of a surface of the article. This may prevent biofilm formation or reduce the prevalence of a biofilm on the surface.

The article may be a component in a medical environment; preferably the article is a component in a hospital or a veterinary hospital.

However, it will be appreciated that the prevention and/or treatment of biofilms can provide benefits in non-medical settings. The article may be an article involved with medicine or surgery, food preparation, personal hygiene or water treatment.

The article may, in one embodiment, be a medical device such as a surgical instrument, or an implant or prosthesis, or a medical machine or component thereof.

Preferably the article is a dressing. The dressing may be adhesive or non-adhesive. The dressing may be, or comprise, gauze, lint, cotton wool, cloth, semi-permeable film (e.g. polyurethane film), semi-permeable foam (e.g. silicone), hydrogel (e.g. containing methacrylate and/or polyvinyl pyrrolidine), hydrocolloid (e.g. containing carboxymethylcellulose, gelatin and/or pectin), hydrofiber (e.g. containing sodium carboxymethyl cellulose) or alginate (e.g. containing sodium and/or calcium alginate). Preferably the dressing is an impregnated dressing, wherein the dressing is impregnated with a compound of Formula (I) or composition thereof, for example a composition comprising a compound of Formula (I) and a retinoid. Hydroxyethylcellulose is a possible additional or alternative component of a hydrocolloid or hydrogel.

Preferably the article is a gel, for example a hydrogel. The hydrogel may be made of natural materials such as collagen (e.g. derived from jellyfish). The hydrogel may contain hydroxyethyl cellulose.

The article (e.g. dressing or other medical device) may be sterile, and may optionally be provided in a sterile package to prevent contamination prior to use of the article.

The article may be an item of Pulmonary Function Testing (PFT) equipment (e.g. a spirometer), a ventilator, a humidifier, a nebulizer, a positive airway pressure (PAP) device, a manual resuscitator, a mask, an item of oxygen equipment, a circuit, a disposable, a respirator, an inhaler, or an inhalation chamber, or a part thereof.

The release of the active ingredient(s) from the article may be regulated to provide a controlled or modified (e.g. slow) release.

The article may be an item of equipment for the treatment of a wound, such as a pressure wound device (e.g. a vacuum-assisted closure (VAC therapy) device or a Chariker-Jeter wound sealing kit), for instance for negative pressure wound therapy (NPWT).

The article may be selected from surgical instruments, for example forceps, reamers, pushers, pliers, or retractors; permanent implants, such as artificial heart valves, voice prostheses, prosthetic joints, implanted artificial lenses, stents (e.g. vascular stents), and shunts (e.g. hydrocephalus shunts); and non-permanent implants, such as pacemakers and pacemaker leads, drain tubes, endotracheal or gastrointestinal tubes, temporary or trial prosthetic joints, surgical pins, guidewires, surgical staples, cannulas, subcutaneous or transcutaneous ports, and indwelling catheters and catheter connectors, and contact lenses. The article may also be a medical machine or component thereof, for example, it may be selected from dialysis machines, dialysis water delivery systems, water circuits within a dialysis unit and water delivery systems for respirator therapy. The implant may be an orthopaedic implant.

In one embodiment the article is a catheter. Examples of indwelling catheters include urinary catheters, vascular catheters (e.g. central venous catheters, dialysis catheters, peripheral venous catheters, arterial catheters and pulmonary artery Swan-Ganz catheters), peritoneal dialysis catheters, central venous catheters and needleless connectors.

The present invention allows the prevention of medical device-associated bacterial infections by providing the polymer that inhibits bacterial attachment on the surface of medical device products.

For example, the polymers of the present invention offer the possibility to effectively reduce catheter-related bacterial infections.

The article may be suitable for use in an industrial setting, such as the hull or propulsion component of a maritime vessel, paper manufacturing apparatus, oil recovery plant apparatus, food processing plant apparatus, water processing plant apparatus, drinking water distribution system apparatus, a cooling tower, or any other article that may be affected by biofouling on surfaces.

In one embodiment, the article is a component of process equipment, such as cooling equipment, water treatment equipment, or food processing equipment. In one such embodiment, the component is of a cooling tower, a water treatment plant, a dairy processing plant, a food processing plant, a chemical process plant, or a pharmaceutical process plant.

The article may comprise a food preparation surface, such as kitchen worktop, cutting board, sink, stove, refrigerator surface, or a bathroom surface, such as toilet, sink, bathtub, shower, or drain.

The article may comprise a floor, door or window surface.

The article may be a toilet bowl, sink, bathtub, drain, high-chair, work surface, food processing machine, cutting board, item of cutlery, dishwasher, duct (e.g. air duct), air conditioning unit, toilet bowl, chamber pot, urinary catheter, dialysis machine, animal feeding device (e.g. trough), animal drinker, milking apparatus, tank (e.g. for milk and/or cheese), pipeline, centrifuge, pasteurizer, packaging tool and/or worksurface, or a component thereof.

### Biofilm-Related Conditions

Biofilms can form on many surfaces, including on and within the tissues of the human or animal body. Biofilms can form on the epidermal layer of the body.

A subject with a biofilm-related condition may be a human or a non-human animal. The subject may be a mammal, such as a primate, dog, cat, cattle, pig, horse or sheep.

Biofilms may form on a surface within the mouth, where they can result in and/or exacerbate conditions such as dental plaque and dental calculus leading to dental decay (dental caries), periodontal disease (such as periodontitis, periapical periodontitis, and peri-implantitis), and gingivitis.

Biofilms may form within the gut, where they can result in and/or exacerbate conditions such as inflammatory bowel disease, colorectal disease and peritonitis.

Biofilms may form on and/or around the genital region (e.g. pubis, vagina and/or penis), where they can result in and/or exacerbate conditions such as urinary tract infections, bacterial vaginosis and vulvovaginal candidiasis.

Biofilms may form within the ear, where they can result in and/or exacerbate conditions such as ear infections, such as otitis media (which may be chronic, suppurative, and/or with effusion), and cholesteatoma.

Biofilms may form within the nose, where they can result in and/or exacerbate conditions such as sinusitis (which may be chronic and/or include rhinosinusitis).

Biofilms may form within the throat, where they can result in and/or exacerbate conditions such as adenotonsillitis (e.g. chronic).

Biofilms may form within the prostate gland, where they can result in and/or exacerbate conditions such as prostatitis.

Biofilms may form within the lungs, where they can result in and/or exacerbate conditions such as pneumonia, bronchitis (e.g. viral bronchitis), and cystic fibrosis.

Biofilms in the lungs may exacerbate chronic obstructive pulmonary disease (COPD, e.g. emphysema and/or chronic bronchitis), (lower) respiratory tract infections, and cystic fibrosis. These can be related to Pseudomonas bacteria, which the compound and composition of the invention have been shown to be effective against.

Biofilms in the lungs may exacerbate acute bronchitis, bronchiolitis, pneumonia, acute exacerbations of chronic obstructive pulmonary disease/chronic bronchitis (AECB), and acute exacerbation of bronchiectasis. The clearing of biofilms and control of bacteria is also useful in the prevention and/or treatment of these conditions.

Biofilms may form within the eye, where they can result in and/or exacerbate conditions such as dry eye, blepharitis syndrome, endophthalmitis, keratitis (including contact lens-associated keratitis), scleral buckle infection, lacrimal system infections and periorbital infections.

Biofilms may form within the heart, where they can result in and/or exacerbate conditions such as endocarditis and myocardial infarctions.

Biofilms may form within the kidney, where they can result in and/or exacerbate conditions such as kidney stones.

Biofilms may form that have an effect within bone, where they can result in and/or exacerbate conditions such as osteomyelitis.

Biofilms may form on the skin, where they can result in and/or exacerbate conditions such as ulcers (e.g. venous ulcers), acne, psoriasis, chronic wounds, hindradenitis suppurativa, atopic dermatitis, candidiasis, and onychomycosis.

Biofilms may form on wounds, e.g. orthopaedic impact, chronic and/or cutaneous wounds, where they can result in and/or exacerbate conditions such as delayed wound healing and necrotising fasciitis.

The biofilm-related condition may generally be an infection. In particular, the infection may be a bacterial infection. The bacteria may be Gram-positive or Gram-negative. For instance, the bacteria may be selected from the list consisting of Staphylococcus aureus, Pseudomonas aeruginosa, Escherichia coli, Proteus mirabilis, Acinetobacter baumannii, Acinetobacter haemolyticus and Streptococcus. The bacteria may be selected from the list consisting of Staphylococcus aureus, Pseudomonas aeruginosa, Escherichia coli, Proteus mirabilis, Acinetobacter baumannii, Acinetobacter haemolyticus, Cutibacterium acnes and Streptococcus. The bacteria may be of a genus or species selected from the list consisting of Enterococcus Faecalis, Staphylococcus Epidermis, Klebsiella, Enterobacteriaceae, Bacteroides, Fusobacterium, Peptostreptococcus, Clostridium, Campylobacter, Proteus mirabilis, P. aerobicus, Enteroccus and Mycobacterium (e.g. M. tuberculosis, M. leprae, M. avium and M. abscessus). The bacteria may be an antimicrobial (e.g. antibiotic) resistant variant of bacteria, such as a bacteria selected from the list above. The infection may be a fungal infection. For instance, the fungi may be of a genus or species selected from the list consisting of Candida, (e.g. Candida albicans, C. parapsilosis, C. tropicalis, C. krusei and C. glabrata, C. parasilosis), Aspergillus (e.g. Aspergillus fumigatus, A. flavus), Cryptococcus (e.g. Cryptococcus neoformans, Cryptococcus gattii), Trichosporon (e.g. Trichosporon asahii), Coccidioides (e.g. Coccidioides immitis, C. posadasii), Pneumocystis, Rhodotorula, Malassezia pachydermatis, Histoplasma capsulatum, Paracoccidioides brasiliensis, Saccharomyces cerevisiae, Mucorales, Blastoschizomyces, Fusarium (e.g. Fusarium oxysporum, F. proliferatum, F. verticillioides), Blastoschizomyces capitatus, Blastomyces dermatitidis, Malassezia pachydermatis, Pneumocystis, Trichosporon asahii, Rhizopus, Rhizomucor, Cladosporium carrionii, Phialophora verrucosa, Fonsecaea pedrosoi. Microsporum, Epidermophytum, Trichophyton, Histoplasma capsulatum, Mucor, Rhizopus, Pneumocystis jirovecii, Sporothrix schenckii, Malassezia furfur (Pityrosporum orbiculare), and M. globose.

The compound of Formula (I) or composition thereof may partially or fully remove the biofilm. There may be conditions where the complete removal of the biofilm is undesirable, for example where an overexpression of a normally beneficial bacterium is causing a biofilm-related condition. Therefore, a controlled reduction in the amount of biofilm may successfully restore a healthy condition in the subject. Such applications are envisaged for oral preventions and/or treatments, e.g. in the prevention and/or treatment of dental plaque.

### Administration

It will be appreciated that the optimal dose may vary depending according to factors such as the presence of other compounds in the composition, the age, weight and condition of the subject to be treated, the type and severity of the condition to be prevented and/or treated and the frequency and route of administration.

The TPGS may be provided or administered as a dose (for example a daily dose) of 1 ng/kg or more, such as 10 ng/kg or more, or 100 ng/kg or more, preferably 1 μm/kg or more, such as 5 μm/kg or more, or 10 μm/kg or more, for example 100 μm/kg or more, or 1 mg/kg or more, such as 5 mg/kg or more, or 10 mg/kg or more, for instance 50 mg/kg or more, or 100 mg/kg or more, such as 500 mg/kg or more, relative to the weight of the subject. The dose may be 10 g/kg or less, such as 5 g/kg or less, or 1 g/kg or less, such as 500 mg/kg or less, or 200 mg/kg or less, for example 100 mg/kg or less, relative to the weight of the subject. The dose may be from 1 ng/kg to 10 g/kg, such as from 100 ng/kg to 5 g/kg, or from 1 μm/kg to 1 g/kg, for example from 5 μm/kg to 200 mg/kg, relative to the weight of the subject. Such doses may be particularly suited to systemic administration.

For topical administration, the TPGS may be provided or administered as a dose (for example a daily dose) of 1 ng/10 cm² or more, such as 10 ng/10 cm² or more, or 100 ng/10 cm² or more, preferably 1 μm/10 cm² or more, such as 5 μm/10 cm² or more, or 10 μm/10 cm² or more, for example 100 μm/10 cm² or more, or 1 mg/10 cm² or more, such as 5 mg/10 cm² or more, or 10 mg/10 cm² or more, for instance 50 mg/10 cm² or more, or 100 mg/10 cm² or more, such as 500 mg/10 cm² or more, relative to the surface area of the subject to which the composition is applied. The dose may be 10 g/10 cm² or less, such as 5 g/10 cm² or less, or 1 g/10 cm² or less, such as 500 mg/10 cm² or less, or 200 mg/10 cm² or less, for example 100 mg/10 cm² or less, relative to the surface area of the subject to which the composition is applied. The dose may be from 1 ng/10 cm² to 10 g/10 cm², such as from 100 ng/10 cm² to 5 g/10 cm², or from 1 μm/10 cm² to 1 g/10 cm², for example from 5 μm/10 cm² to 200 mg/10 cm², relative to the surface area of the subject to which the composition is applied.

The compound or composition thereof may be administered by any suitable means to the site of the biofilm that requires prevention and/or treatment. Preferably the compound or composition is administered by topical (e.g. epicutaneous) administration, for example in the prevention and/or treatment of infections such as in a wound.

When the composition is administered by topical administration and comprises a bio-active agent, the composition may comprise the bio-active agent (e.g. a retinoid such as retinol) in a concentration of 50 μm/ml or less, such as 20 μm/ml or less, or 10 μm/ml or less, such as 8 μm/ml or less, preferably 6 μm/ml or less. The concentration may be 0.1 μm/ml or more, such as 0.5 μm/ml or more, or 1 μm/ml, for example 2 μm/ml or more, or 4 μm/ml or more. The concentration may be from 0.1 to 50 μm/ml, such as from 1 to 10 μm/ml. Such concentrations have been found to provide the beneficial effects of the bio-active agent in relation to secondary healing, but without being toxic to the dermis layer of the skin (at around 50 μm depth).

The compound or composition thereof may be administered to the skin, mouth, gut, eye, urethra, genital region, nose, ear, throat, lungs, prostate, kidney, heart and/or kidney, for example a wound thereof. Preferably the compound or composition thereof is administered to a wound of the skin, for example the epidermis (around 30 μm depth). The wound may be infected, for example chronically infected. Urethral administration may be used to prevent and/or treat a UTL Administration by inhalation may be used to prevent and/or treat lung-related conditions such as COPD and cystic fibrosis.

Alternatively or additionally, the composition may be administered by injection, for example intramuscular and/or (preferably) intraarticular injection, which may allow the composition to infuse at a controlled and/or modified rate.

The composition may be administered by other systemic means, such as intravenous injection and/or orally. Intravenous injection may be controlled or modified by using a drip or syringe driver. Oral administration may be controlled or modified by providing the composition in a controlled/modified release formulation, for example wax.

### EXAMPLES

Staphylococcus aureus (NCTC12493; MRSA) and Pseudomonas aeruginosa (NCTC12903) were stored in Pro-Lab Diagnostics Microbank (Fisher, Basingstoke UK) tubes at −80° C. before streaking onto Nutrient Agar (Oxoid, Basingstoke, UK) and incubated at 37° C., aerobically for 18 hours.

The two strains of bacteria were produced using different media, with no flocculation occurring. S. aureus was produced using Dulbecco's Modified Eagle Medium and took five to seven days to grow a mature biofilm. P. aeruginosa was produced using M9 minimal media and 1% glucose media and took three days to grow a mature biofilm. Alternatively, Luria-Bertani (LB) broth could be used to grow P. aeruginosa.

### Preparation of Micelles of TPGS

Micellar compositions of TPGS may be prepared following the method of GB2550346, i.e. by dissolving any hydrophobic compounds (e.g. a retinoid such as retinol) in an organic solvent (e.g. ethanol) to provide a hydrophobic phase; adding the hydrophobic phase into aqueous TPGS; and removing organic solvent (e.g. in vacuo). Additionally or alternatively, methods to prepare micelles described in WO 2017/194965 A1 may be used.

Alternatively, micellar compositions of TPGS may be prepared following the protocol set out below.

Prewarmed 20 wt % TPGS in aqueous solution (20 mL) was mixed with 0.6 wt % retinol solution in ethanol (5 mL). The mixture was maintained at 50° C. and homogenised at 5,000 rpm (Fisher brand 850 Homogeniser, P/N:15505819 & probe specification: diameter: 10 mm, length: 115 mm, bottom type: sawtooth, material: stainless steel) for 30 min until the total volume was reduced to 20 mL to obtain a formulation containing 0.6 wt % retinol micelles; 0.1 wt % polyhexanide solution is added to the pre-formed retinol micelles to achieve a formulation of 0.3 wt % retinol and 0.1 wt % PHMB micelles.

This protocol has been found to reduce large bulk materials into nano size droplets resulting in uniform particle size with improved stability.

After homogenization, the formulations were allowed to cool to ambient temperature and thereafter transferred into scintillation vials and stored in the dark.

Micellar compositions of TPGS may be prepared by the following method:
Provide an aqueous solution of TPGS as previously e.g. 20% aqueous TPGS;
Add 0.21 g retinol to 3 g alcohol (e.g. ethanol) to provide a solution of approx. 7 wt % retinol;
Add 3 g of the retinol solution to 35 g of the TPGS solution (approx. 7 g TPGS) to provide a composition comprising 0.21 g retinol, 7 g TPGS, 3 g alcohol and 28 g water (total weight approx. 38.21 g; per 100 g, the composition contains approximately: 18.34 g TPGS, 0.55 g retinol, 7.85 g alcohol, 83.28 g water); and
Diluting 100 g of the composition 1:1 with water (to a total volume of 200 g) provides approximately, per 100 g, 10 g TPGS, 0.3 g retinol, and 4.28 g alcohol.

The mixture may be homogenised as noted above before or after the dilution step.

### Raman Characterisation of Compositions

The laser Raman spectroscopy was employed to determine the physiochemical changes between the individual compounds and the formulations. In particular, the Raman spectra of each of 0.3% retinol and 10% TPGS were compared with the Raman spectrum of a formulation of both 10% TPGS with 0.3% retinol. Raman shifts observed between 800 and 850 cm⁻¹ and between 1,500 and 1,650 cm⁻¹ changes in the Raman environments of the individual compounds and the formulation. The observed changes in the shifts of the Raman spectra for the formulation compared to the individual compounds could be due to the strong π-electron interactions in the formulation.

### MIC Assays

Minimum inhibitory concentrations (MICs) were determined using the broth microdilution method (EUCAST, Clinical Microbiology and Infection (2003), 9(8)). The inoculated liquid culture (grown dynamically overnight at 37° C. in MHB) was adjusted to obtain a ~0.1 OD at 600 nm, equivalent to a 0.5 McFarland Standard. A range of concentrations (diluted in MHB) for each of the compounds were prepared in 96-well plates, followed by the addition of the inoculated media. The plates were then incubated statically for 18 hours at 37° C. Following the incubation period, the lowest concentration of compound presenting an inhibition of bacterial growth was labelled the MIC. The MIC was either determined through visual observation or using a TECAN, OD₆₀₀ where the OD<50% of the media-only control.

The MIC of a range of compositions were determined using the NCCLS broth microdilution method, against S. aureus and P. aeruginosa. The compounds tested were:
Retinol—as 100 mg/mL (10%) in ethanol
Curcumin—as 4 mg/mL (0.4%) in ethanol
PHMB—as 20% in water
D-α-Tocopherol polyethylene glycol 1000 succinate (TPGS)—as 200 mg/mL (20%) in Milli-Q water
Polysorbate 40—pure
Ethanol—pure

The results are shown in the table below:

| Compound | | Bacterial strain | | | |
|---|---|---|---|---|---|
| (concentration | | S. aureus | | P. aeruginosa | |
| before inoculation) | Vehicle | w/v % (mg/mL) | | w/v % (mg/mL) | |
| Retinol | Ethanol | 5 | (50) | 5 | (50) |
| Curcumin | Ethanol | 0.2 | (2) | 0.2 | (2) |
| Curcumin | TPGS | 0.0125 | | 0.1 | |
| PHMB | Water | 0.000625 | | 0.005 | |
| TPGS | Water | 20 | (200) | 20 | (200) |

| Ethanol | — | 6.25 | 6.25 |
|---|---|---|---|
| Polysorbate 40 | — | >25% | >25% |

TPGS demonstrated inhibition of S. aureus growth at 10% v/v, and P. aeruginosa growth at 20% v/v.

PHMB showed the highest inhibitory activity against both bacterial strains, preventing growth at concentrations of 0.000625% and 0.005% v/v for S. aureus and P. aeruginosa, respectively.

Curcumin solubilised in TPGS showed the next highest inhibitory activity against S. aureus at 0.0125% w/v, and P. aeruginosa at 0.1% w/v.

Curcumin solubilised in ethanol showed a concentration of 0.2% w/v for inhibition of bacterial growth. This was much greater than ethanol alone which had a minimum inhibitory concentration of 6.25% for both strains, suggesting a major contribution of curcumin to bacteria growth inhibition. Retinol solubilised in ethanol provided a minimum inhibitory concentration of 5% w/v against both pathogens, slightly higher than that of ethanol. Polysorbate 40 showed no inhibitory activity at 25% (w/v).

These data show that TPGS, and compositions thereof (e.g. compounds including bio-active agents such as curcumin) surprisingly provide some antimicrobial activity against bacteria such as S. aureus and P. aeruginosa.

### Crystal Violet Biofilm Clearance Assays

CV was used to stain biofilms and hence determine the degree of biofilm growth from the intensity of the stain. The biofilm growth method and visualisation/analysis techniques were followed from J. Merritt et al. (Curr Protoc Microbiol. (2005); Chapter 1:Unit 1B.1), with modifications. S. aureus (NCTC12493) was grown in DMEM and P. aeruginosa (NCTC12903) was grown in M9 minimal media+1% glucose. Inoculated liquid culture was diluted in 1:100 in their respective medias. Into 96-well plates, 100 μL of the inoculant was added to each well. Following this, the plate was covered with a lid and incubated at 37° C. for 24-hour intervals of up to 7 days, aerobically. The compounds were added and incubated at 37° C. for a further 24 hours. Planktonic bacteria were removed using a pipette, and the biofilms were washed with Milli-Q water. To each well, 125 μL of 0.1% crystal violet (CV) solution was added and left at room temperature for 10-15 minutes. After this, the 0.1% CV solution was removed, and the well was washed until the waste ran clear. The stain was left to air-dry, before 200 μL 30% acetic acid was added to each well for 15 minutes on a plate shaker. To a new 96-well plate, 125 μL stained biofilm solution was transferred and the optical density was read at 600 nm. A comparison was made between the uninhibited biofilm and any biofilm reductions capabilities of the added compounds.

The following compounds were tested: 0.000625% PHMB, 10% TPGS, 10% empty micelles of TPGS (EM) and 6.25% ethanol (EtOH). The candidate compounds were each, therefore, applied at their respective MIC values.

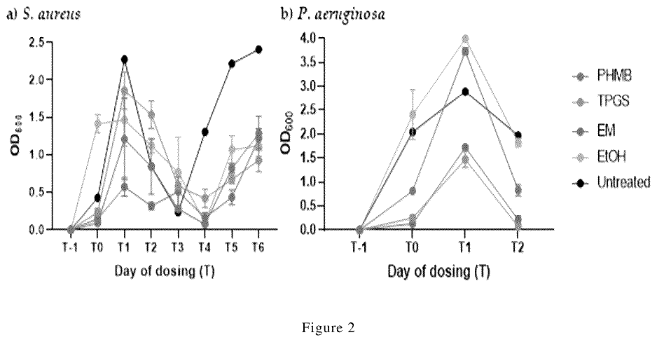

FIG. 2 of the accompanying drawings shows the results of this study into the amount of present after treatment with the compounds at different time intervals.

The results show that TPGS generally decreases the amount of biofilm present compared to the untreated control. This is shown most clearly in P. aeruginosa, where TPGS was the most effective compound. TPGS also shows a reduction in the amount of biofilm present for S. aureus, when dosed on day 0, day 1 or from day 4 onwards.

Therefore, TPGS can be used as an effective treatment against biofilms.

EM (empty micelles of TPGS) provided even better activity against the biofilms than TPGS in non-micellar form for both S. aureus and P. aeruginosa. This improved anti-biofilm activity was hypothesised to be due to the organised micelle formation of the TPGS.

PHMB showed good biofilm clearance in S. aureus. However, for P. aeruginosa, there is an increase in biofilm when dosed on T1. EtOH has little to no effect on P. aeruginosa; dosing at day T1 increased biofilm growth. In the case of S. aureus, at TO there was an increase in biofilm when dosed with EtOH. This was hypothesised to be due to the bacteria attempting to protect itself. Over the course of 7 days, EtOH in some cases increases or decreases the biofilm. All other compounds cleared some of the biofilm.

### Combination Studies

The amount of biofilm remaining and the CFU count following treatment after a certain time period with 0.1% PHMB, 10% TPGS or the combination of 0.1% PHMB and 10% TPGS were determined.

Bacterial growth kinetics of S. aureus grown in DMEM and P. aeruginosa grown in M9 minimal media+1% glucose were determined. The amount of biofilm remaining was determined using the CV staining method as described above.

CFU count was determined by the following method: The inoculated liquid culture (grown dynamically overnight at 37° C. in liquid growth media) was spun down in a centrifuged (at max (13.3) speed for 5 minutes) (reference). The supernatant was removed and resuspend in PBS (1:100 dilution). Into each well, 20 μL of inoculated culture LB broth was added to 180 μL LB broth. The antiseptic candidate PHMB was added to each well, 1 μL of 20% PHMB, resulting in an overall concentration of 0.1%. For the untreated wells, 1 μL of PBS was added. The 96-well plate was placed in a TECAN measuring at OD600 over 24 hours at 15-minute intervals. Resulting growth kinetic experiments were conducted using mid-logarithmically growing cultures. S. aureus (NCTC12493) was grown in Dulbecco's Modified Eagle's Medium (DMEM), and (b) P. aeruginosa (NCTC12903) was grown in Luria-Bertani (LB) following the dosing of antiseptic candidate PHMB (0.1%) at 3 hours indicated by the red arrow. Following this, the wells were homogenised, and 20 μL samples were plate spotted onto nutrient agar (NA). These plates were incubated for 18 hours at 37° C. before colonies were counted.

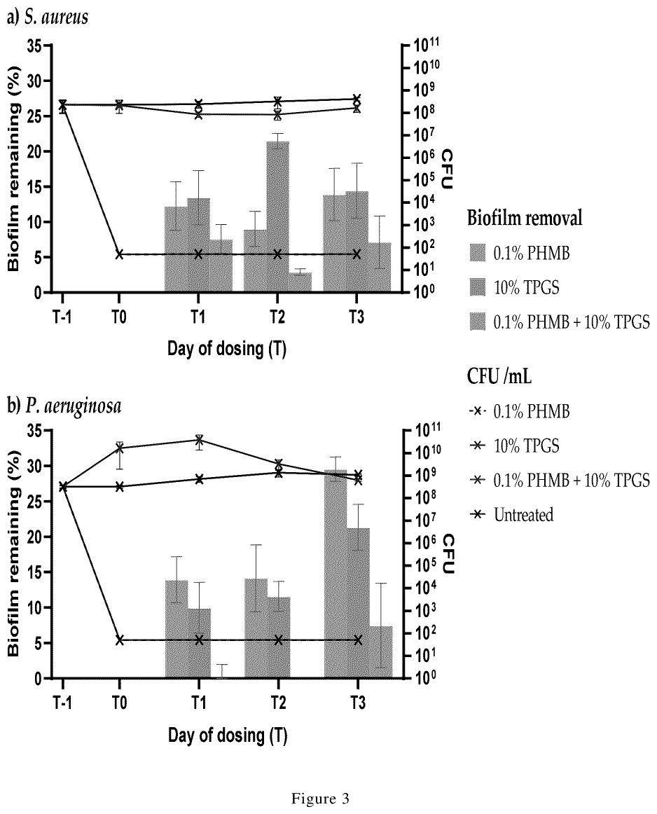

FIG. 3 of the accompanying drawings shows the results of these studies into the CFU and amount of biofilm remaining.

The percentage biofilm remaining graphs show that all three treatments (0.1% PHMB alone, 0.1% PHMB with 10% TPGS, and 10% TPGS alone) were effective in fully reducing the biofilm remaining when treated at day 0.

Additionally, all three compositions were effective at significantly reducing the amount of biofilm remaining when treatment was undertaken at day 1, 2 or 3. Specifically, treatment was effective with 0.1% PHMB alone or with 10% TPGS alone.

However, when treatment was performed with the combination of 0.1% PHMB and 10% TPGS, there was a step change in the efficacy of the treatment. The biofilm was significantly reduced.

The difference when using the combination treatment was most significant for P. aeruginosa, where no biofilm remained after the treatments performed on days T1 and T2. Treatment on day T3 provided a significant difference even when compared to either of the other treatments.

In relation to the CFU analysis, when the biofilms were not treated the CFU of the bacteria remained relatively constant. The CFU of the bacteria also remained substantially constant when treated with 10% TPGS alone. This is not surprising, as TPGS is more effective at breaking up biofilms than being an antimicrobial agent. However, when treated with 0.1% PHMB with 10% TPGS, the CFU count completely reduced when treated on any day.

This demonstrates the surprising antimicrobial effect of the combination of TPGS with a bio-active agent, such as an antimicrobial agent—in this case, PHMB.

Therefore, the combination of TPGS and an antimicrobial agent, such as PHMB, not only provides a surprising increased effect against the amount of biofilm remaining after treatment, but also provides significant effects against the viability of the bacterial cells.

### Wound Healing Investigations

Samples of 3D human skin wound model (organotypic 3D FT-SWM, EpoDermFT 400, MatTek®, organotypic skin equivalents, which include small epidermal only wounds induced using a 3 mm punch biopsy) were treated with one of the aqueous compositions listed below for 6 days and were subsequently histologically examined. Specifically, cultures of the EpiDermFT-400 cells (MatTek®) in 6-well tissue culture plates were maintained and assayed in a humidified atmosphere incubator (Thermo Fischer®) with ~5% CO₂ and a temperature of 37° C. Five antibiotics-free formulations and 1 placebo were tested against the cultures of the EpiDermFT-400 cells in 6-well tissue culture plates in a sterile environment. The 3D wound model consisted of keratinocytes and fibroblasts (EpiDermFT-400) located in the epidermis and dermis, respectively. The diameter of the epidermis and dermis of the model were punched by the manufacturer with a 3 mm biopsy punch to create a 3 mm in diameter wound. The wounded dermis was filled with collagen fibres for migration of the fibroblast into the defect. The six test formulations (25 μL) were topically applied against the wound in the apical compartment of the EpiDermFT-400 tissue supported with 2.5 mL assay media in the basal compartment. The treated cells were maintained in a humidified atmosphere incubator with ~5% CO₂ and a temperature of 37° C. for 6 days. Thereafter, they were stored in a 10% formalin aqueous solution for histological analysis.

The inner wound diameters of the treated and control organotypic wound models were measured at 30/60 m and 80 m, representing largely to epidermis and dermis, respectively.

The samples were treated with one of the following compositions:
C) phosphate-buffered saline
M) 10% TPGS as micelles (i.e. empty micelles)
F1) 10% TPGS as micelles and 0.3% retinol
F2) 10% TPGS as micelles and 1% retinol
F3) 10% TPGS as micelles, 0.3% retinol and 0.1% PHMB
F4) 10% TPGS as micelles and 0.1% PHMB

For histological analysis, the treated organotypic 3D FT-SWM (EpiDermFT-400) were gently removed from the membrane inserts and evaluated grossly. Then, they were processed routinely, embedded in paraffin-wax, and blocks were created making emphasis on flattening the surface of the treated EpiDermFT-400 tissue specimens to obtain homogeneous histological sections. Furthermore, 5 μm-thick sections were obtained at depths of 30/60 μm and 80 μm and were stained with haematoxylin and eosin. Sections obtained were scanned in a calibrated NanoZoomer Digital slide scanner (Hamamatsu®, Hertfordshire, UK). Digital measurements of the inner diameters of the skin samples were taken at 30/60 μm-depth (largely epidermis) and 80 μm-depth (largely dermis).
Upon gross examination, it was observed that punch wounds were not necessarily in the centre of the specimens and superficial growth and total thickness was slightly variable amongst specimens. The results for model wound contraction are shown in the table below:

| | | 3D wound Sample | | | |
|---|---|---|---|---|---|
| | | 30/60 μm-depth | | 80 μm-depth | |
| | | Diameter | | | |
| Name | Formulation | mm | % WD | mm | % WD |
| C | PBS | 2.55 | 15.00 | 2.76 | 8.00 |
| M | 10% TPGS | 2.45 | 18.33 | 2.23 | 25.67 |
| F1 | 10% TPGS; | 1.26 | 58.00 | 0.68 | 77.33 |
| | 0.3% Retinol | | | | |
| F2 | 10% TPGS; | 2.18 | 27.33 | 2.35 | 21.67 |
| | 1% Retinol | | | | |
| F3 | 10% TPGS; 0.3% | 2.29 | 23.67 | 2.43 | 19.00 |
| | Retinol; 0.1% PHMB | | | | |
| F4 | 10% TPGS; | 2.67 | 11.00 | 2.44 | 18.67 |
| | 0.1% PHMB | | | | |

It was observed that the inner wound diameter in the presence of the formulations ranged from 1.26 to 2.67 mm at 30/60 μm depth, in comparison to 2.55 mm in the control. Among all the formulations, F1 containing 0.3% retinol micelles demonstrated the highest contraction. F4 containing 0.1% PHMB micelle showed the least wound contraction. F1 and F2 had similar compositions except for a higher dose of retinol in the latter, however, this did not translate to improved contraction as the former showed better outcome. Comparison of F3, containing 0.3% retinol and 0.1% PHMB micelles, to F4, 0.1% PHMB with no retinol, there was an improvement in the wound contraction for F3. Moreover, the empty TPGS micelles (M) and PBS (control) demonstrated inner wound diameter of 2.45 mm and 2.55 mm, respectively. It was also found that all the treated specimens (F1-F4) except M had variable degrees of demonstrable migration of epithelial cells towards the edges of the punch wound at 30/60 m depth. This shows that including a retinoid, such as retinol, can increase the rate of wound healing.

Although epithelial migration was observed in the specimen treated with M at the depth of 10 jm, it seemed to be limited at 30/60 m depth.

The inner wound diameter measurements taken at the depth of 80 μm demonstrated a similar trend in wound contraction to the pattern observed at 30/60 μm depth. This was surprising given the increased difficulty of penetrating further into the skin, and the difference in tissues observed at 80 μm depth of skin (i.e. dermis) compared to 30/60 μm (i.e. epidermis).

The formulations lead to the inner wound diameter at 80 μm ranging from 0.68 to 2.44 mm compared to the control showing 2.76 mm. Among all the formulations, F1 exhibited the best wound recovery with inner wound diameter of 0.68 mm compared to the control of 2.76 mm. F4 showed the least contraction with inner wound diameter of 2.44 mm compared to the rest of the formulations, but it still performed better than the control.

This shows the benefits of compositions comprising compounds of Formula (I). Good wound closure performance was observed, particularly where the compositions also comprised retinol.

Furthermore, all samples that contained retinol or PHMB as well as TPGS micelles had variable degrees of demonstrable migration of epithelial cells towards the edges of the punch wound at 30/60 μm-depth.

This shows that deeper wound treatment can be achieved by using bio-active agents, especially retinoids, in combination with the TPGS micelles.

Therefore, based on this wound model, the compounds of the invention can be successfully used to treat wounds.

### Ex-Vivo Model (Pig Skin)

Skin penetration studies were performed on dermatomed porcine skin, dosed using vertical Franz diffusion cells.

A scalpel was used to cut each Franz cell disc into a smaller portion for sectioning, and was sanitised with ethanol between samples. A fresh cryostat blade was used for each sample. The cryostat temperature was set to −33° C., and samples equilibrated in the cryostat for approximately 45 minutes prior to sectioning.

A sample composition of the invention comprising retinol and TPGS was applied to the outer surface of a sample of pig skin for 24 hours. After 24 hours, the skin surface was cleaned and cross-sections 30 μm thick were prepared using cryosectioning and mounted onto glass coverslips. SRS (stimulated raman spectroscopy) imaging was used to observe the penetration of the composition through the skin.

SRS imaging used a Leica SP8 microscope with SRS and CARS. A PicoEmerald-S laser system output two pulsed 2 ps laser beams (a 1031 nm Stokes beam spatially and temporally overlapped with a tuneable pump beam). The Stokes beam was modulated at 20 MHz and stimulated Raman loss signals were detected in transmission using a lock-in amplifier (UHFLI, Zurich instruments). CARS and SHG/fluorescence signals were epi-detected in separate PMT channels. A water immersion×25 magnification lens (0.95 NA, Leica) was used. A short working distance air condenser (0.9 NA, Leica) was used. Laser power was 30%, which corresponded to approximately 10 mW for the pump beam and 30 mW for the Stokes beam at the sample. Leica LAS-X version 3.5.5.19976 acquisition software was used.

Retinol was observed down to depths of 60 μm through the skin, i.e. in the epidermis. This indicates excellent penetration of retinol when in the presence of TPGS. A similar experiment using a composition including PHMB (1 wt %) did not show penetration of the retinol.

### In Vitro Activity/Monitoring—Fibroblast Cells

Fibroblasts were exposed to compositions containing 0.31 μg/mL or 0.25 μg/mL, and a control sample with no retinol, over a period of 7 days. SRS-SHG microscopy was used to determine absorption of the retinol by the fibroblasts at days 1, 3 and 7 after exposure.

The SRS-SHG microscopy showed concentration and time dependent response of the fibroblasts to the retinol. In particular, outside of nuclei, increasing SHG signals were detected with increasing concentration of retinol and with the time since the beginning of the exposure.

This illustrates the benefits of modified (e.g. slow) release compositions of retinol and/or enabling retinol to penetrate deep into the skin (e.g. 30 or 50 μm).

This also demonstrates vitamin A (e.g. retinol) activating fibroblasts. The combination of SHG and SRS helps visualise collagen. Collagen fibres have a very suitable structure for generating SHG signal. Fibrillar collagen is highly anisotropic and the SHG signal generated is coherently amplified because of the tight alignment of repeating structures within the collagen triple helix and within fibrils. Therefore, SHG and SRS can be used to visualise collagen.

Collagen proteins help maintain the structural framework of tissues and have an important role in healing wounds. The compositions of the invention also enable the rate of collagen expression to be controlled by adjusting the vitamin A or retinol dose delivery and hence the speed of expression. Fibrosis is generated in a wound by accelerated fibroblast proliferation and collagen expression. Managing the rate of fibroblast proliferation and activation, and thus collagen expression, can minimise fibrosis for healthy tissue healing and potentially, as fibrotic tissue is renewed in the same process as non-fibrotic tissue, reverse fibrosis and fibrotic tissue generation. Scars such as hypertrophic and keloid are generated by accelerated or excess fibroblast. Some scars such as pitted and acne scars can be a result of insufficient collagen generation and controlled increasing fibroblast proliferation, and collagen generation can benefit pitted scar prevention or recovery. Thus, controlling fibroblasts can reduce scar formation (or the appearance thereof).

### In Vitro Penetration Testing of 0.3% Retinol Samples

The retinol recovery of formulations of the present invention on a Strat-M membrane (synthetic, non-animal based model for transdermal diffusion testing) were tested against alternative compositions that are currently available on the market, and quantified using HPLC. The applied dose of retinol in each case was 500 μg/cm². The results are presented in the table below.

| | | Retinol Recovery | |
|---|---|---|---|
| | | measured on | Retinol |
| | | Strat-M | Recovery |
| Sample | | membrane | (% applied |
| ID | Sample Description | (μg/cm²) | dose) |
| S1 | 0.3% TPGS micellar retinol | 20.53 | 4.11 |
| | solution diluted in H₂O | | |
| S2 | 0.3% Competitor retinol cream | 90.04 | 18.01 |
| S3 | 0.3% TPGS micellar retinol | 15.29 | 3.02 |
| | diluted in 0.3% w/v xanthan | | |
| | gum (XG) | | |
| S4 | 0.3% Competitor retinol cream | 93.14 | 18.42 |
| | (repeat of S2) | | |

The data in the above table demonstrates that retinol recovery from the surface of the skin for samples S1 and S3 (invention) are much lower than that of the competitor's product (S2, S4-comparative). This indicates that the penetration of retinol into the Strat-M membrane is more effective using the composition of the present invention than in the competitor's product.

Furthermore, the similar results of samples S1 and S3 shows that thickening agents such as XG can be tolerated without affecting retinol absorption.

### Cytotoxicity

The cytotoxicity of compositions in HaCaT cells (keratinocytes) was determined using the WST 1 assay to determine percentage cell viability. The compositions tested were A) TPGS at 3, 5 and 7% by volume, B) combinations of TPGS and PHMB in amounts of 3%/0.03%, 5%/0.05%, 7%/0.07% and 10%/0.1% respectively, by volume, and C) combinations of TPGS and retinol in amounts of 3%/0.03%, 5%/0.05%, 7%/0.10% and 10%/0.3% respectively, by volume. The cell viability represents the fraction of active cells after treatment and incubation for 24 h expressed as a normalised percentage.

TPGS alone left more than 50% viable cells, even at the highest concentration tested. TPGS in combination with retinol or PHMB left at least 50% viable cells at 7% TPGS and 0.07% PHMB, while more than 50% viable cells also remained for TPGS (10%) and retinol (0.3%).

Therefore, in addition to the anti-biofilm and antimicrobial effects shown above, the compositions of the invention are not significantly cytotoxic.

### Measurement of Hydrodynamic Particle Size

Hydrodynamic particle size was determined by performing DLS measurements using a Beckman Coulter sub-micron particle analyser instrument at 20° C.

The average (mean) particle size (diameter) of TPGS was determined as being 8.82 nm with a polydispersity index (PI) of 0.047.

1. A method of treatment, prevention or treatment of a biofilm-related condition, the method comprising administrating a compound of Formula (I) 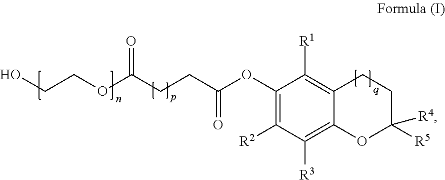
wherein:
R¹, R², R³ and R⁴ are each independently H or a C1-6 hydrocarbon group that is optionally substituted by one or more Y group;
R⁵ is a C6-30 hydrocarbon group;
n is from 3 to 100;
p is from 0 to 6;
q is from 0 to 3; and
Y is selected from the list consisting of ether, ester, sulfone, sulfoxide, amide, amine, boronate ester, ketone and aldehyde.

2. The method of claim 1, wherein the composition comprises aqueous micelles of the compound of Formula (I).

3. (canceled)

4. The method of claim 2, wherein the micelles have an average diameter of from 8 to 30 nm.

5. The method of claim 1, wherein the composition is for topical administration to the skin.

6. The method of claim 1, wherein the composition further comprises a retinoid, polyhexamethylene biguanide (PHMB) and/or curcumin.

7. The method of claim 1, wherein the composition is a gel.

8. The method of claim 1, wherein the composition includes micelles of the compound of formula (I) in an amount of up to 10 wt %, and wherein the micelle size is up to 30 nm.

9. The method of claim 8, wherein the composition further includes PHMB in an amount of up to 0.1 wt % and/or a retinoid in an amount of up to 0.3 wt %.

10. The method of claim 9, wherein the compound of Formula (I) is tocopheryl polyethylene glycol succinate (TPGS), and the composition includes the TPGS in an amount of 10 wt %, and the composition includes the retinoid, and the retinoid is retinol, and the composition includes the retinol in an amount of 0.3 wt %.

11. (canceled)

12. The method of claim 1, wherein the composition comprises the compound of Formula (I) in an amount of 5% or more by weight.

13. The method of claim 1, wherein the biofilm-related condition is selected from the list consisting of: dental plaque, dental calculus, dental decay, periodontal disease, gingivitis, inflammatory bowel disease, colorectal disease, peritonitis, urinary tract infection, bacterial vaginosis, vulvovaginal candidiasis, ear infection, cholesteatoma, sinusitis, adenotonsillitis, prostatitis, pneumonia, bronchitis, COPD, cystic fibrosis, dry eye, blepharitis syndrome, endophthalmitis, keratitis, scleral buckle infection, lacrimal system infection, periorbital infection, endocarditis, myocardial infarction, kidney stones, osteomyelitis, ulcers, acne, psoriasis, hindradenitis suppurativa, atopic dermatitis, candidiasis, onychomycosis, chronic wound and/or non-healing wound, orthopaedic wound, orthopaedic impact wound, cutaneous wound, surgical wound, necrotising fasciitis and infection.

14. The method of claim 1, wherein the composition is suitable for administration by inhalation.

15. The method of claim 1, wherein:
R¹, R², R³ and R⁴ are each independently H or a C1-3 alkyl group;
R⁵ is a C6-30 hydrocarbon group;
n is from 10 to 40;
p is from 0 to 2; and
q is 0 or 1.

16. The method of claim 1, wherein the Formula (I) is represented by Formula (II): 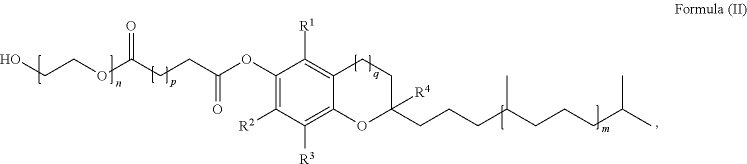
wherein m of the Formula (II) is an integer from 0 to 4.

17. The method of claim 1, wherein the compound is tocopheryl polyethylene glycol succinate.

18. The method of claim 1, wherein the composition is in the form of a gel.

19. (canceled)

20. An article comprising a compound of Formula (I), or a composition thereof 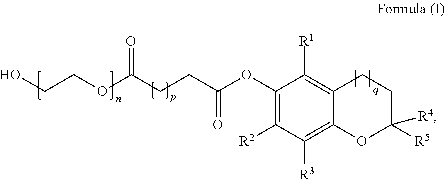
wherein:
R¹, R², R³ and R⁴ are each independently H or a C1-6 hydrocarbon group that is optionally substituted by one or more Y group;
R⁵ is a C6-30 hydrocarbon group;
n is from 3 to 100;
p is from 0 to 6;
q is from 0 to 3; and
Y is selected from the list consisting of ether, ester, sulfone, sulfoxide, amide, amine, boronate ester, ketone and aldehyde.

21. (canceled)

22. The article of claim 20, wherein the article is a catheter, an implant, or a wound dressing.

23. The article of claim 20, wherein the article is Pulmonary Function Testing equipment, a ventilator, a humidifier, a nebulizer, a positive airway pressure device, a manual resuscitator, a mask, an item of oxygen equipment, a circuit, a disposable, a respirator, an inhaler, or an inhalation chamber, a nebuliser, or a part thereof.

24. A method comprising the steps of:
providing a compound of Formula (I) or a composition thereof 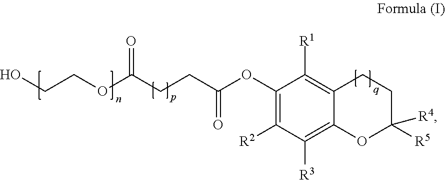
wherein:
R¹, R², R³ and R⁴ are each independently H or a C1-6 hydrocarbon group that is optionally substituted by one or more Y group;
R⁵ is a C6-30 hydrocarbon group;
n is from 3 to 100;
p is from 0 to 6;
q is from 0 to 3; and
Y is selected from the list consisting of ether, ester, sulfone, sulfoxide, amide, amine, boronate ester, ketone and aldehyde,
and
applying the compound or the composition thereof to a surface.

25. (canceled)

26. (canceled)
